(19) United States
(12) Patent Application Publication
(10) Pub. No.: US 2022/0016082 A1
(43) Pub. Date: Jan. 20, 2022

(54) COMBINATION THERAPIES FOR HIGH AND VERY HIGH RISK MDS

(71) Applicant: MEI Pharma, Inc., San Diego, CA (US)

(72) Inventor: Richard GHALIE, San Diego, CA (US)

(21) Appl. No.: 17/294,513

(22) PCT Filed: Nov. 26, 2019
(86) PCT No.: PCT/US2019/063360

§ 371 Date: May 17, 2021

Related U.S. Application Data
(60) Provisional application No. 62/773,490, filed on Nov. 30, 2018.

(51) Int. Cl. A61K 31/4184; A61K 31/706; A61P 35/02

(52) U.S. Cl. CPC A61K 31/4184; A61P 35/02; A61K 31/706

(57) ABSTRACT

Provided herein are methods of treating high and very high risk MDS comprising administering pracinostat and a DNA hypomethylating agent. S

### CROSS-REFERENCE

This patent application claims the benefit of U.S. Provisional Application No. 62/773,490, filed Nov. 30, 2018 which is incorporated herein by reference in its entirety.

### BACKGROUND OF THE INVENTION

Local chromatin architecture is generally recognized as an important factor in the regulation of gene expression. The architecture of chromatin, a protein-DNA complex, is strongly influenced by post-translational modifications of the histones which are the protein components. Reversible acetylation of histones is a key component in the regulation of gene expression by altering the accessibility of transcription factors to DNA. In general, increased levels of histone acetylation are associated with increased transcriptional activity, whereas decreased levels of acetylation are associated with repression of gene expression. In normal cells, histone deacetylases (HDACs) and histone acetyltransferase together control the level of acetylation of histones to maintain a balance. Inhibition of HDACs results in the accumulation of acetylated histones, which results in a variety of cell type dependent cellular responses, such as apoptosis, necrosis, differentiation, cell survival, inhibition of proliferation and cytostasis.

Inhibitors of HDAC have been studied for their therapeutic effects on cancer cells. For example, suberoylanilide hydroxamic acid (SAHA) is a potent inducer of differentiation and/or apoptosis in murine erythroleukemia, bladder, and myeloma cell lines. SAHA has been shown to suppress the growth of prostate cancer cells in vitro and in vivo. Other inhibitors of HDAC that have been widely studied for their anti-cancer activities are trichostatin A (TSA) and trapoxin B. Trichostatin A is a reversible inhibitor of mammalian HDAC. Trapoxin B is a cyclic tetrapeptide, which is an irreversible inhibitor of mammalian HDAC. However, due to the in vivo instability of these compounds they are less desirable as anti-cancer drugs. The in vivo activity of recently disclosed inhibitors can be directly monitored by their ability to increase the amount of acetylated histones in the biological sample. HDAC inhibitors have been reported to interfere with neurodegenerative processes, for instance, HDAC inhibitors arrest polyglutamine-dependent neurodegeneration. In addition, HDAC inhibitors have also been known to inhibit production of cytokines such as TNF, IFN, IL-1 which are known to be implicated in inflammatory diseases and/or immune system disorders.

Nevertheless, there is still a need to provide further HDAC inhibitor combinations that would be expected to have useful, improved pharmaceutical properties in the treatment of diseases such as cancer, neurodegenerative diseases, disorders involving angiogenesis and inflammatory and/or immune system disorders.

### SUMMARY OF THE INVENTION

Disclosed herein is a method of treating high or very high risk myelodysplastic syndromes (MDS) in a patient in need thereof, the method comprising administering to the patient:

(i) a DNA hypomethylating agent; and

(ii) about 45 mg of a compound of formula (I):

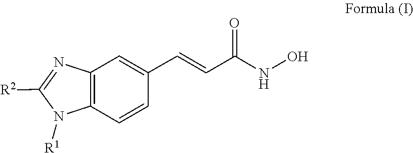

wherein
R¹ is —(CR²⁰R²¹)ₘ—(CR²²R²³)ₙ—(CR²⁴R²⁵)ₒ—NR²⁶R²⁷;
R² is alkyl, fluoroalkyl, cyano, C₂-C₆alkenyl, C₂-C₆alkynyl, or heteroalkyl optionally substituted with ═O;
each R²⁰, R²¹, R²², R²³, R²⁴, and R²⁵ is independently H or methyl;
each R²⁶ and R²⁷ is independently H, hydroxyalkyl, or alkyl; and
m, n, and o are independently integers of 0, 1, 2, 3, or 4;
or a pharmaceutically acceptable salt, isotopic variant, or prodrug thereof.

In some embodiments of a method of treating high or very high risk myelodysplastic syndromes (MDS), the compound of formula (I) has the structure:

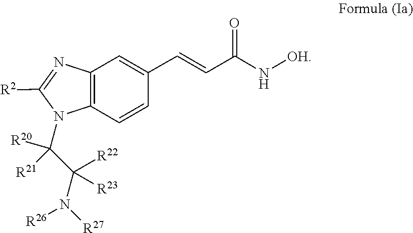

In some embodiments of a method of treating high or very high risk myelodysplastic syndromes (MDS), R²⁶ and R²⁷ are independently H or alkyl.

In some embodiments of a method of treating high or very high risk myelodysplastic syndromes (MDS), R²⁶ and R²⁷ are independently H, methyl, ethyl, isopropyl, propyl, butyl, isobutyl, pentyl, hexyl or heptyl.

In some embodiments of a method of treating high or very high risk myelodysplastic syndromes (MDS), R¹ has the structure

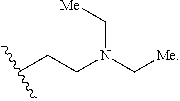 [SMILES: *CCN(CC)CC]

In some embodiments of a method of treating high or very high risk myelodysplastic syndromes (MDS), R² is ethyl, 1-methyl-ethyl, 2,2,2-trifluoroethyl, propyl, 2-methyl-propyl, 2,2-dimethyl-propyl, 3,3,3-trifluoro-propyl, butyl, 3,3-dimethyl-butyl, pentyl, 2,4,4-trimethyl-pentyl, hexyl or octyl.

In some embodiments of a method of treating high or very high risk myelodysplastic syndromes (MDS), R² is butyl.

In some embodiments of a method of treating high or very high risk myelodysplastic syndromes (MDS), the compound of formula (I) is pracinostat:

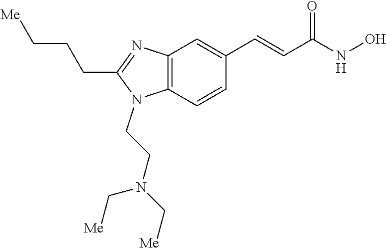 [SMILES: CCCCc1nc2cc(/C=C/C(=O)NO)ccc2n1CCN(CC)CC]

or a pharmaceutically acceptable salt, isotopic variant, or prodrug thereof.

In some embodiments of a method of treating high or very high risk myelodysplastic syndromes (MDS), the DNA hypomethylating agent is 5-azacytidine (azacitidine), 5-azadeoxycytidine (decitabine), SGI-110, zebularine, or procaine.

In some embodiments of a method of treating high or very high risk myelodysplastic syndromes (MDS), the DNA hypomethylating agent is 5-azacytidine (azacitidine).

In some embodiments of a method of treating high or very high risk myelodysplastic syndromes (MDS), the DNA hypomethylating agent is 5-azadeoxycytidine (decitabine).

In some embodiments of a method of treating high or very high risk myelodysplastic syndromes (MDS), the method is for treating high risk myelodysplastic syndromes (MDS).

In some embodiments of a method of treating high or very high risk myelodysplastic syndromes (MDS), the method is for treating very high risk myelodysplastic syndromes (MDS).

In some embodiments of a method of treating high or very high risk myelodysplastic syndromes (MDS), the patient in need thereof has not been previously treated with a DNA hypomethylating agent.

In some embodiments of a method of treating high or very high risk myelodysplastic syndromes (MDS), the patient in need thereof has been previously treated with transfusions, hematopoietic growth factors, or immunosuppressive therapy.

In some embodiments of a method of treating high or very high risk myelodysplastic syndromes (MDS), the MDS is refractory, non-responsive, or resistant to chemotherapy and/or haploidentical stem cell transplantation.

In some embodiments of a method of treating high or very high risk myelodysplastic syndromes (MDS), the DNA hypomethylating agent is administered in an amount from about 5 mg/m² to about 125 mg/m².

In some embodiments of a method of treating high or very high risk myelodysplastic syndromes (MDS), the DNA hypomethylating agent is administered in an amount of about 75 mg/m².

In some embodiments of a method of treating high or very high risk myelodysplastic syndromes (MDS), the compound of formula (I), or a pharmaceutically acceptable salt, isotopic variant, or prodrug thereof, is administered orally and the hypomethylating agent is administered intravenously or subcutaneously.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered in cycles of 28 days.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 3 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 4 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 5 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 6 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 7 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 8 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 9 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered for at least 10 cycles.

In some embodiments of a method of treating high or very high risk myelodysplastic syndromes (MDS), the compound of formula (I), or a pharmaceutically acceptable salt, isotopic variant, or prodrug thereof, and the DNA hypomethylating agent are administered until complete remission (CR) is observed.

In some embodiments of a method of treating high or very high risk myelodysplastic syndromes (MDS), the compound of formula (I), or a pharmaceutically acceptable salt, isotopic variant, or prodrug thereof, is administered for 3 days each week for 3 consecutive weeks, followed by 1 week of rest of each 28-day cycle.

In some embodiments of a method of treating high or very high risk myelodysplastic syndromes (MDS), the method further comprises administering the compound of formula (I), or a pharmaceutically acceptable salt, isotopic variant, or prodrug thereof, for 3 days each week for 2 consecutive weeks, followed by 2 weeks of rest of each 28-day cycle.

In some embodiments of a method of treating high or very high risk myelodysplastic syndromes (MDS), the DNA hypomethylating agent is administered for 7 days of each 28-day cycle.

In some embodiments of a method of treating high or very high risk myelodysplastic syndromes (MDS), the DNA hypomethylating agent is administered on a 5-2-2 schedule: DNA hypomethylating agent for 5 consecutive days followed by 2 days of rest, followed by DNA hypomethylating agent for 2 consecutive days of each 28-day cycle.

In some embodiments of a method of treating high or very high risk myelodysplastic syndromes (MDS), the discontinuation rate due to adverse events is less than 25%, less than 20%, less than 15%, less than 10%, less than 8%, less than 5%.

In some embodiments of a method of treating high or very high risk myelodysplastic syndromes (MDS), the discontinuation rate due to adverse events is about 4%.

In some embodiments of a method of treating high or very high risk myelodysplastic syndromes (MDS), the adverse event is selected from constipation, nausea, fatigue, decreased appetite, diarrhea, edema peripheral, hypoalbuminemia, dyspnea, hypokalemia, vomiting, dizziness, febrile neutropenia, anemia, neutropenia, and thrombocytopenia.

Also disclosed herein is a method of treating high or very high risk myelodysplastic syndromes (MDS) in a patient in need thereof, the method comprising administering to the patient:
(i) a DNA hypomethylating agent; and
(ii) about 45 mg of pracinostat, or a pharmaceutically acceptable salt, isotopic variant, or prodrug thereof, wherein pracinostat is orally administered to the patient 3 days each week for 3 consecutive weeks, followed by 1 week of rest, in 28-day cycles.

In some embodiments of a method of treating high or very high risk myelodysplastic syndromes (MDS), the DNA hypomethylating agent and pracinostat or a pharmaceutically acceptable salt, isotopic variant, or prodrug thereof, are administered for at least 3 cycles.

In some embodiments of a method of treating high or very high risk myelodysplastic syndromes (MDS), the DNA hypomethylating agent and pracinostat or a pharmaceutically acceptable salt, isotopic variant, or prodrug thereof, are administered for at least 4 cycles.

In some embodiments of a method of treating high or very high risk myelodysplastic syndromes (MDS), the DNA hypomethylating agent and pracinostat or a pharmaceutically acceptable salt, isotopic variant, or prodrug thereof, are administered for at least 5 cycles.

In some embodiments of a method of treating high or very high risk myelodysplastic syndromes (MDS), the DNA hypomethylating agent and pracinostat or a pharmaceutically acceptable salt, isotopic variant, or prodrug thereof, are administered for at least 6 cycles.

In some embodiments of a method of treating high or very high risk myelodysplastic syndromes (MDS), the DNA hypomethylating agent and pracinostat or a pharmaceutically acceptable salt, isotopic variant, or prodrug thereof, are administered for at least 3 cycles, followed by further administering pracinostat, or a pharmaceutically acceptable salt, isotopic variant, or prodrug thereof, for 3 days each week for 2 consecutive weeks, followed by 2 weeks of rest of each 28-day cycle for at least 1 cycle.

Also disclosed herein is a method of treating high or very high risk myelodysplastic syndromes (MDS) in a patient in need thereof, the method comprising administering to the patient:
(i) about 75 mg/m² of 5-azacytidine (azacitidine), wherein 5-azacytidine (azacitidine) is administered intravenously or subcutaneously for 7 days of each 28-day cycle; and
(ii) about 45 mg of pracinostat, or a pharmaceutically acceptable salt, isotopic variant, or prodrug thereof, wherein pracinostat is orally administered for 3 days each week for 3 consecutive weeks, followed by 1 week of rest, in 28-day cycles.

In some embodiments of a method of treating high or very high risk myelodysplastic syndromes (MDS), 5-azacytidine (azacitidine) and pracinostat or a pharmaceutically acceptable salt, isotopic variant, or prodrug thereof, are administered for at least 3 cycles.

In some embodiments of a method of treating high or very high risk myelodysplastic syndromes (MDS), 5-azacytidine (azacitidine) and pracinostat or a pharmaceutically acceptable salt, isotopic variant, or prodrug thereof, are administered for at least 4 cycles.

In some embodiments of a method of treating high or very high risk myelodysplastic syndromes (MDS), 5-azacytidine (azacitidine) and pracinostat or a pharmaceutically acceptable salt, isotopic variant, or prodrug thereof, are administered for at least 5 cycles.

In some embodiments of a method of treating high or very high risk myelodysplastic syndromes (MDS), 5-azacytidine (azacitidine) and pracinostat or a pharmaceutically acceptable salt, isotopic variant, or prodrug thereof, are administered for at least 6 cycles.

In some embodiments of a method of treating high or very high risk myelodysplastic syndromes (MDS), 5-azacytidine (azacitidine) and pracinostat or a pharmaceutically acceptable salt, isotopic variant, or prodrug thereof, are administered for at least 3 cycles, followed by further administering pracinostat, or a pharmaceutically acceptable salt, isotopic variant, or prodrug thereof, for 3 days each week for 2 consecutive weeks, followed by 2 weeks of rest of each 28-day cycle for at least 1 cycle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows cumulative survival probability following treatment with pracinostat and azacitidine.

FIG. 2 shows overall survival comparisons between azacitidine (AZA) monotherapy and AZA in combination with lenalidomide (LEN) or vorinostat (VOR).

FIG. 3 shows the overall survival following treatment with azacitidine or conventional care: best supportive care, low-dose cytarabine, or intensive chemotherapy.

FIG. 4 shows the overall survival following treatment with panobinostat and azacitidine treatment in MDS and AML.

FIG. 5 shows the overall survival following treatment with azacitidine with or without entinostat in patients with patients with myelodysplastic syndrome and chronic myelomonocytic leukemia.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

There is a continuing need to develop and provide effective therapies for the treatment of disease and disorders associated with dysregulation of histone deacetylase (e.g., cancer). Described herein in some embodiments is a combination therapy for treating cancer.

### Certain Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

Unless otherwise noted, terminology used herein should be given its normal meaning as understood by one of skill in the art.

As used herein, the term unsubstituted means that there is no substituent or that the only substituents are hydrogen.

The term “optionally substituted” as used throughout the specification denotes that the group may or may not be further substituted or fused (so as to form a condensed polycyclic system), with one or more substituent groups. Preferably the substituent groups are one or more groups independently selected from the group consisting of halogen, ═O, ═S, —CN, —NO₂, —CF₃, —OCF₃, alkyl, alkenyl, alkynyl, haloalkyl, haloalkenyl, haloalkynyl, heteroalkyl, cycloalkyl, cycloalkenyl, heterocycloalkyl, heterocycloalkenyl, aryl, heteroaryl, cycloalkylalkyl, heterocycloalkylalkyl, heteroarylalkyl, arylalkyl, cycloalkylalkenyl, heterocycloalkylalkenyl, arylalkenyl, heteroarylalkenyl, cycloalkylheteroalkyl, heterocycloalkylheteroalkyl, arylheteroalkyl, heteroarylheteroalkyl, hydroxy, hydroxyalkyl, alkoxy, alkoxyalkyl, alkoxycycloalkyl, alkoxyheterocycloalkyl, alkoxyaryl, alkoxyheteroaryl, alkoxycarbonyl, alkylaminocarbonyl, alkenyloxy, alkynyloxy, cycloalkyloxy, cycloalkenyloxy, heterocycloalkyloxy, heterocycloalkenyloxy, aryloxy, phenoxy, benzyloxy, heteroaryloxy, arylalkyloxy, arylalkyl, heteroarylalkyl, cycloalkylalkyl, heterocycloalkylalkyl, arylalkyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, alkylsulfinyl, arylsulfinyl, aminosulfinylaminoalkyl, —COOH, —COR⁵, —C(O)OR⁵, CONHR⁵, NHCOR⁵, NHCOOR⁵, NHCONHR⁵, C(═NOH)R⁵, —SH, —SR⁵, —OR⁵, and acyl.

“Alkyl” as a group or part of a group refers to a straight or branched aliphatic hydrocarbon group, preferably a C₁-C₁₄ alkyl, more preferably C₁-C₁₀ alkyl, preferably C₁-C₆ or C₁-C₃ unless otherwise noted. Examples of suitable straight and branched C₁-C₆ alkyl substituents include methyl, ethyl, n-propyl, 2-propyl, n-butyl, sec-butyl, t-butyl, hexyl, and the like. The group may be a terminal group or a bridging group.

“Alkylamino” includes both monoalkylamino and dialkylamino, unless specified. “Monoalkylamino” means a —NH-Alkyl group, in which alkyl is as defined above. “Dialkylamino” means a —N(alkyl)₂ group, in which each alkyl may be the same or different and are each as defined herein for alkyl. The alkyl group is preferably a C₁-C₆ alkyl group. The group may be a terminal group or a bridging group.

“Arylamino” includes both mono-arylamino and di-arylamino unless specified. Mono-arylamino means a group of formula aryl NH—, in which aryl is as defined herein. di-arylamino means a group of formula (aryl₂)N— where each aryl may be the same or different and are each as defined herein for aryl. The group may be a terminal group or a bridging group.

“Acyl” means an alkyl-CO— group in which the alkyl group is as described herein. Examples of acyl include acetyl and benzoyl. The alkyl group is preferably a C₁-C₆ alkyl group. The group may be a terminal group or a bridging group.

“Alkenyl” as a group or part of a group denotes an aliphatic hydrocarbon group containing at least one carbon-carbon double bond and which may be straight or branched preferably having 2-14 carbon atoms, more preferably 2-12 carbon atoms, most preferably 2-6 carbon atoms, in the normal chain. The group may contain a plurality of double bonds in the normal chain and the orientation about each is independently E or Z. Exemplary alkenyl groups include, but are not limited to, ethenyl, propenyl, butenyl, pentenyl, hexenyl, heptenyl, octenyl and nonenyl. The group may be a terminal group or a bridging group.

“Alkoxy” refers to an —O-alkyl group in which alkyl is defined herein. Preferably the alkoxy is a C₁-C₆alkoxy. Examples include, but are not limited to, methoxy and ethoxy. The group may be a terminal group or a bridging group.

“Alkenyloxy” refers to an —O— alkenyl group in which alkenyl is as defined herein. Preferred alkenyloxy groups are C₁-C₆ alkenyloxy groups. The group may be a terminal group or a bridging group.

“Alkynyloxy” refers to an —O-alkynyl group in which alkynyl is as defined herein. Preferred alkynyloxy groups are C₁-C₆ alkynyloxy groups. The group may be a terminal group or a bridging group.

“Alkoxycarbonyl” refers to an —C(O)—O-alkyl group in which alkyl is as defined herein. The alkyl group is preferably a C₁-C₆ alkyl group. Examples include, but not limited to, methoxycarbonyl and ethoxycarbonyl. The group may be a terminal group or a bridging group.

“Akylsulfinyl” means a —S(O)-alkyl group in which alkyl is as defined above. The alkyl group is preferably a C₁-C₆ alkyl group. Exemplary alkylsulfinyl groups include, but not limited to, methylsulfinyl and ethylsulfinyl. The group may be a terminal group or a bridging group.

“Alkylsulfonyl” refers to a —S(O)₂-alkyl group in which alkyl is as defined above. The alkyl group is preferably a C₁-C₆ alkyl group. Examples include, but not limited to methylsulfonyl and ethylsulfonyl. The group may be a terminal group or a bridging group.

“Alkynyl as a group or part of a group means an aliphatic hydrocarbon group containing a carbon-carbon triple bond and which may be straight or branched preferably having from 2-14 carbon atoms, more preferably 2-12 carbon atoms, more preferably 2-6 carbon atoms in the normal chain. Exemplary structures include, but are not limited to, ethynyl and propynyl. The group may be a terminal group or a bridging group.

“Alkylaminocarbonyl” refers to an alkylamino-carbonyl group in which alkylamino is as defined above. The group may be a terminal group or a bridging group.

“Cycloalkyl” refers to a saturated or partially saturated, monocyclic or fused or spiro polycyclic, carbocycle preferably containing from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

“Cycloalkenyl” means a non-aromatic monocyclic or multicyclic ring system containing at least one carbon-carbon double bond and preferably having from 5-10 carbon atoms per ring. Exemplary monocyclic cycloalkenyl rings include cycloheptenyl, cyclohexenyl or cycloheptenyl. The cycloalkenyl group may be substituted by one or more substituent groups. The group may be a terminal group or a bridging group.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

“Cycloalkylalkyl” means a cycloalkyl-alkyl- group in which the cycloalkyl and alkyl moieties are as previously described. Exemplary monocycloalkylalkyl groups include cyclopropylmethyl, cyclopentylmethyl, cyclohexylmethyl and cycloheptylmethyl. The group may be a terminal group or a bridging group.

“Halogen” represents chlorine, fluorine, bromine or iodine.

“Heterocycloalkyl” refers to a saturated or partially saturated monocyclic, bicyclic, or polycyclic ring containing at least one heteroatom selected from nitrogen, sulfur, oxygen, preferably from 1 to 3 heteroatoms in at least one ring. Each ring is preferably from 3 to 10 membered, more preferably 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. The group may be a terminal group or a bridging group.

“Heterocycloalkenyl” refers to a heterocycloalkyl as described above but containing at least one double bond. The group may be a terminal group or a bridging group.

“Heterocycloalkylalkyl” refers to a heterocycloalkyl-alkyl group in which the heterocycloalkyl and alkyl moieties are as previously described. Exemplary heterocycloalkylalkyl groups include (2-tetrahydrofuryl)methyl, (2-tetrahydrothiofuranyl)methyl. The group may be a terminal group or a bridging group.

“Heteroalkyl” refers to a straight- or branched-chain alkyl group preferably having from 2 to 14 atoms, more preferably 2 to 10 atoms in the chain, one or more of which is a heteroatom selected from S, O, and N. Exemplary heteroalkyls include alkyl ethers, secondary and tertiary alkyl amines, alkyl sulfides, and the like. The group may be a terminal group or a bridging group.

“Aryl” as a group or part of a group denotes (i) an optionally substituted monocyclic, or fused polycyclic, aromatic carbocycle (ring structure having ring atoms that are all carbon) preferably having from 5 to 12 atoms per ring. Examples of aryl groups include phenyl, naphthyl, and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Arylalkenyl” means an aryl-alkenyl- group in which the aryl and alkenyl are as previously described. Exemplary arylalkenyl groups include phenylallyl. The group may be a terminal group or a bridging group.

“Arylalkyl” means an aryl-alkyl- group in which the aryl and alkyl moieties are as previously described. Preferred arylalkyl groups contain a C₁₋₅ alkyl moiety. Exemplary arylalkyl groups include benzyl, phenethyl and naphthelenemethyl. The group may be a terminal group or a bridging group.

“Arylacyl” means an aryl-acyl- group in which the aryl and acyl moieties are as previously described. In general the aryl moiety is attached to the alkyl portion of the acyl moiety, typically to the terminal carbon of the alkyl portion of the acyl moiety. Preferred arylacyl groups contain a C₁-C₅ alkyl moiety in the acyl moiety. Exemplary arylacyl groups include 2-phenyl-acetyl. The group may be a terminal group or a bridging group.

“Heteroaryl” either alone or part of a group refers to groups containing an aromatic ring (preferably a 5 or 6 membered aromatic ring) having one or more heteroatoms as ring atoms in the aromatic ring with the remainder of the ring atoms being carbon atoms. Suitable heteroatoms include nitrogen, oxygen and sulfur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl, 1-, 2-, or 3-indolyl, and 2-, or 3-thienyl. The group may be a terminal group or a bridging group.

“Heteroarylalkyl” means a heteroaryl-alkyl group in which the heteroaryl and alkyl moieties are as previously described. Preferred heteroarylalkyl groups contain a lower alkyl moiety. Exemplary heteroarylalkyl groups include pyridylmethyl. The group may be a terminal group or a bridging group.

“Lower alkyl” as a group means unless otherwise specified, an aliphatic hydrocarbon group which may be straight or branched having 1 to 6 carbon atoms in the chain, more preferably 1 to 4 carbons such as methyl, ethyl, propyl (n-propyl or isopropyl) or butyl (n-butyl, isobutyl or tertiary-butyl). The group may be a terminal group or a bridging group.

In Formula (I), as well as in Formulae (1a)-(If) defining sub-sets of compounds within Formula (I), there is shown a benzimidazole ring system. Within this ring system, there are substitutable positions at the 4-, 5-, 6-, and 7-ring positions. In each of Formulae (I), (Ia), and (Ib), there is a requirement for attachment of an acidic moiety at one of the ring positions. This acidic moiety may be provided by but is not limited to groups containing, a hydroxamic acid or salt derivatives of such acid which when hydrolyzed would provide the acidic moiety. In some embodiments the acidic moiety may be attached to the ring position through an alkylene group such as —CH₂— or —CH₂—CH₂—, or an alkenylene group such as —CH═CH—. Preferred positions for attachment of the acidic moiety are the 5- and 6-ring positions.

It is understood that included in the family of compounds of Formula (I) are isomeric forms including diastereoisomers, enantiomers, tautomers, and geometrical isomers in “E” or “Z” configurational isomer or a mixture of E and Z isomers. It is also understood that some isomeric forms such as diastereomers, enantiomers, and geometrical isomers can be separated by physical and/or chemical methods and by those skilled in the art.

Some of the compounds of the disclosed embodiments may exist as single stereoisomers, racemates, and/or mixtures of enantiomers and for diastereomers. All such single stereoisomers, racemates and mixtures thereof are intended to be within the scope of the subject matter described and claimed.

Additionally, Formula (I) is intended to cover, where applicable, solvated as well as unsolvated forms of the compounds. Thus, each formula includes compounds having the indicated structure, including the hydrated as well as the non-hydrated forms.

In addition to compounds of the Formula (I), the HDAC inhibiting agents of the various embodiments include pharmaceutically acceptable salts, prodrugs, and active metabolites of such compounds, and pharmaceutically acceptable salts of such metabolites.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic, arylsulfonic. Suitable pharmaceutically acceptable base addition salts of compounds of Formula (I) include metallic salts made from lithium, sodium, potassium, magnesium, calcium, aluminum, and zinc, and organic salts made from organic bases such as choline, diethanolamine, morpholine. Other examples of organic salts are: ammonium salts, quaternary salts such as tetramethylammonium salt; amino acid addition salts such as salts with glycine and arginine. Additional information on pharmaceutically acceptable salts can be found in Remington's Pharmaceutical Sciences, 19^{th} Edition, Mack Publishing Co., Easton, Pa. 1995. In the case of agents that are solids, it is understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of formula (I). For example an ester prodrug of a compound of formula (I) containing a hydroxyl group may be convertible by hydrolysis in vivo to the parent molecule. Suitable esters of compounds of formula (I) containing a hydroxyl group, are for example acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsuiphamates and quinates. As another example an ester prodrug of a compound of formula (I) containing a carboxy group may be convertible by hydrolysis in vivo to the parent molecule. (Examples of ester prodrugs are those described by F. J. Leinweber, Drug Metab. Res., 18:379, 1987).

The term “therapeutically effective amount” or “effective amount” is an amount sufficient to effect beneficial or desired results. An effective amount can be administered in one or more administrations. An effective amount is typically sufficient to palliate, ameliorate, stabilize, reverse, slow or delay the progression of the disease state. A therapeutically effective amount can be readily determined by an attending diagnostician by the use of conventional techniques and by observing results obtained under analogous circumstances. In determining the therapeutically effective amount a number of factors are to be considered including but not limited to, the species of animal, its size, age and general health, the specific condition involved, the severity of the condition, the response of the patient to treatment, the particular compound administered, the mode of administration, the bioavailability of the preparation administered, the dose regime selected, the use of other medications and other relevant circumstances.

The “discontinuation rate” is defined as the number of patients or subjects who discontinue the study drugs prior to the study completion divided by the number of patients or subjects treated.

“Complete remission” or “CR” is achieved when the following criteria are met (responses must last at least 28 days):
Bone marrow: ≤5% myeloblasts with normal maturation of all cell lines
Persistent dysplasia will be noted
Peripheral blood (Hemoglobin≥11 g/dL, Platelets≥100×10⁹/L, Neutrophils≥1.0×10⁹/L and Blasts 0%)

“Partial remission” or “PR” is achieved when the following criteria are met (responses must last at least 28 days): all CR criteria if abnormal before treatment except bone marrow blasts decreased by ≥50% over pretreatment but still >5%.

“Stable disease” is achieved when failure to achieve at least PR, but no evidence of progression for >8 weeks.

“Marrow complete remission” or “Marrow CR” is achieved when the following criteria are met (responses must last at least 28 days):
Bone marrow: ≤5% myeloblasts and decrease by ≥50% over pretreatment
Peripheral blood: if HI (hematological improvement) responses,

“Disease progression” is assessed as follow:
For patients with: Less than 5% blasts: ≥50% increase in blasts to >5% blasts
For patients with: 5%-10% blasts: ≥50% increase to >10% blasts
For patients with: 10%-20% blasts: ≥50% increase to >20% blasts
For patients with: 20%-30% blasts: ≥50% increase to >30% blasts
At least 50% decrement from maximum remission/response in granulocytes or platelets
Reduction in hemoglobin by ≥2 g/dL
Transfusion dependence.

“Hematological improvements” are defined as follow:

Modified IWG Response Criteria for Hematological Improvement

| Hematological | Response Criteria |
|---|---|
| Improvement* | (responses must last at least 8 weeks)† |
| Erythroid response | Hemoglobin increase by ≥1.5 g/dL |
| (pretreatment, <11 | Relevant reduction of units of RBC |
| g/dL) (HI-E) | transfusions by an absolute number of at |
| | least 4 RBC transfusions/8 weeks compared |
| | with the pretreatment transfusion number in |
| | the previous 8 weeks. Only RBC transfusions |
| | given for a hemoglobin of ≤9.0 g/dL |
| | pretreatment will count in the RBC |
| | transfusion response evaluation† |
| Platelet response | Absolute increase of ≥30 × 10⁹/L |
| (pretreatment, <100 × | for patients starting with >20 × 10⁹/L |
| 10⁹/L) (HI-P) | platelets |
| | Increase from <20 × 10⁹/L to >20 × |
| | 10⁹/L and by at least 100%† |
| Neutrophil response | At least 100% increase and an absolute |
| (pretreatment, <1.0 × | increase >0.5 × 10⁹/L† |
| 10⁹/L) (HI-N) | |
| Progression or | At least 1 of the following: |
| relapse after HI‡ | At least 50% decrement from maximum |
| | response levels in granulocytes or platelets |
| | Reduction in hemoglobin by ≥1.5 g/dL |
| | Transfusion dependence |

RBC: Red Blood Cell

### HDAC Inhibiting Agents

In one aspect the present invention provides a compound of the formula (I):

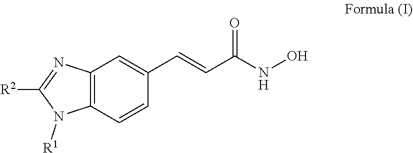

wherein
R is a group having the formula:

—(CR²⁰R²¹)ₘ—(CR²²R²³)ₙ—(CR²⁴R²⁵)ₒ—NR²⁶R²⁷;
R² is alkyl, fluoroalkyl, cyano, C₂-C₆alkenyl, C₂-C₆alkynyl, or heteroalkyl optionally substituted with ═O;
each R²⁰, R²¹, R²², R²³, R²⁴, and R²⁵ is independently H or methyl;
each R²⁶ and R²⁷ is independently H, hydroxyalkyl, or alkyl; and
m, n, and o are independently integers of 0, 1, 2, 3, or 4;
or a pharmaceutically acceptable salt or prodrug thereof.

In some embodiments, the compound of formula (I) has the structure of formula (Ia) or (Ib):

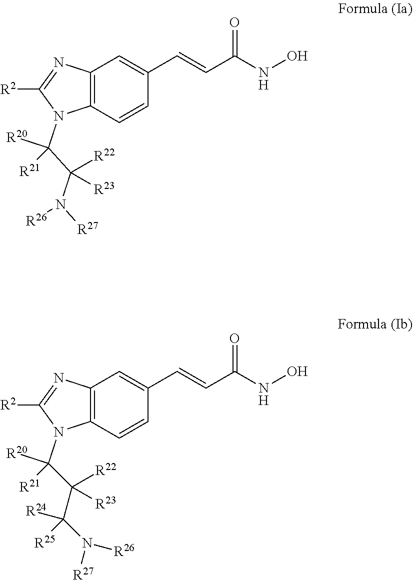

wherein
R² is alkyl, fluoroalkyl, cyano, C₂-C₆alkenyl, C₂-C₆alkynyl, or heteroalkyl optionally substituted with ═O;
each R²⁰, R²¹, R²², R²³, R²⁴, and R²⁵ is independently H or methyl; and
each R²⁶ and R²⁷ is independently H, hydroxyalkyl, or alkyl;
or a pharmaceutically acceptable salt or prodrug thereof.

In each of the above embodiments of the invention R²⁰ and R²¹ may represent a number of different variables. In one embodiment R²⁰ and R²¹ are independently selected from the group consisting of H, alkyl, alkenyl and alkynyl. In another embodiment R²⁰ and R²¹ are independently selected from the group consisting of H and alkyl. In yet another embodiment R²⁰ and R²¹ are independently selected from the group consisting of H, methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, pent-4-enyl, hexyl, heptyl and octyl. In a specific embodiment R²⁰ and R²¹ are both H.

In each of the above embodiments of the invention R²² and R²³ may represent a number of different variables. In one embodiment R²² and R²³ are independently selected from the group consisting of H, alkyl, alkenyl and alkynyl. In another embodiment R²² and R²³ are independently selected from the group consisting of H and alkyl. In yet another embodiment R²² and R²³ are independently selected from the group consisting of H, methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, pent-4-enyl, hexyl, heptyl and octyl. In a further embodiment R²² and R²³ are independently selected from the group consisting of alkyl. In a most specific embodiment R²² and R²³ are both methyl.

In each of the above embodiments of the invention R²⁴ and R²⁵ may represent a number of different variables. In one embodiment R²⁴ and R²⁵ are preferably independently selected from the group consisting of H, alkyl, alkenyl and alkynyl. In another embodiment R²⁴ and R²⁵ are independently selected from the group consisting of H and alkyl. In yet another embodiment R²⁴ and R²⁵ are independently selected from the group consisting of H, methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, pent-4-enyl, hexyl, heptyl and octyl. In a specific embodiment R²⁴ and R²⁵ are both H.

In each of the above embodiments there are a number of values for R²⁶ and R²⁷. In one embodiment R²⁶ and R²⁷ are independently selected from the group consisting of H, alkyl, alkenyl, alkynyl, alkoxyalkyl, and acyl. In another embodiment R²⁶ and R²⁷ are independently selected from the group consisting of H, alkyl and acyl. In a further embodiment R²⁶ and R²⁷ are independently selected from the group consisting of H, methyl, ethyl, isopropyl, propyl, 2-ethyl-propyl, 3,3-dimethyl-propyl, butyl, isobutyl, 3,3-dimethyl-butyl, 2-ethyl-butyl, pentyl, 2-methyl, pentyl, pent-4-enyl, hexyl, heptyl, octyl, acetyl and 2-methoxy-ethyl.

In one specific embodiment R¹ is a group of formula:

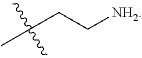 [SMILES: *CCN]

In another specific embodiment R¹ is a group of formula:

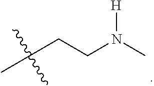 [SMILES: *CCNC]

In another specific embodiment R¹ is a group of formula:

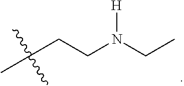 [SMILES: *CCNCC]

In yet another specific embodiment R¹ is a group of formula:

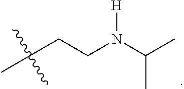 [SMILES: *CCNC(C)C]

In another specific embodiment R¹ is a group of formula:

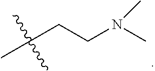 [SMILES: *CCN(C)C]

In another specific embodiment R¹ is a group of formula:

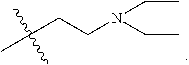 [SMILES: *CCN(CC)CC]

In another specific embodiment R¹ is a group of formula:

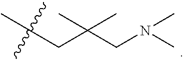 [SMILES: *CC(C)(C)CN(C)C]

In another specific embodiment R¹ is a group of formula:

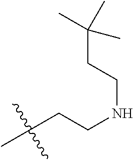 [SMILES: *CCNCCC(C)(C)C]

In another specific embodiment R¹ is a group of formula:

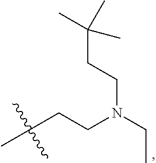 [SMILES: *CCN(CC)CCC(C)(C)C]

In one form of this embodiment R² is alkyl. In one embodiment the alkyl is a C₁-C₁₀ alkyl. In another form of this embodiment the alkyl is a C₁-C₆ alkyl group. In another form of this embodiment R² is selected from the group consisting of methyl; ethyl; propyl; 2-methyl-propyl, 2-2-dimethyl-propyl; isopropyl; 3,3,3-triflouro-propyl; butyl; isobutyl; 3,3-dimethyl-butyl; pentyl; 2,4,4-trimethyl-pentyl; hexyl; heptyl, octyl, nonyl, and 2-methoxy nonyl.

In one form of this embodiment R² is alkenyl. In one form of this embodiment the alkenyl is a C₁-C₁₀ alkenyl. In another form of this embodiment the alkenyl is a C₁-C₆ alkenyl group. In another form of this embodiment R² is selected from the group consisting of ethenyl, prop-1-enyl, prop-2-enyl, but-1-enyl, but-2-enyl but-3-enyl, pent-1-enyl, pent-2-enyl, pent-3-enyl, pent-4-enyl, hex-1-enyl, hex-2-enyl, hex-3-enyl, hex-4-enyl and hex-5-enyl.

In a further embodiment the optional substituents are selected from the group consisting of halogen, ═O, ═S, —CN, —NO₂, alkyl, alkenyl, heteroalkyl, haloalkyl, alkynyl, aryl, cycloalkyl, heterocycloalkyl, heteroaryl, hydroxy, hydroxyalkyl, alkoxy, alkylamino, aminoalkyl, acylamino, phenoxy, alkoxyalkyl, benzyloxy, alkylsulfonyl, arylsulfonyl, aminosulfonyl, —C(O)OR⁵, COOH, SH, and acyl.

In some embodiments, the compound of formula (I) is pracinostat:

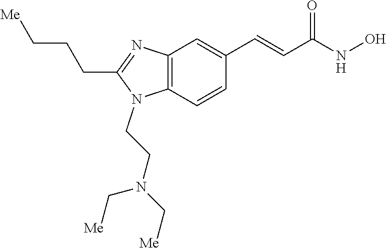 [SMILES: CCCCc1nc2cc(/C=C/C(=O)NO)ccc2n1CCN(CC)CC]

or a pharmaceutically acceptable salt, isotopic variant, or prodrug thereof. Pracinostat may be in a crystalline polymorphic form such as Form 3 or any of those described in international application No. PCT/US2017/030414, published as WO 2017/192451 and entitled “POLYMORPHIC FORMS OF 3-[2-BUTYL-1-(2-DIETHYLAMINO-EHTYL)-IH-BENZOIMIDAZOL-5-YL]-N-HYDROXY-ACRYLAMIDE AND USES THEREOF,” which is incorporated herein in its entirety by reference.

In some embodiments, the compound of formula (I) is:

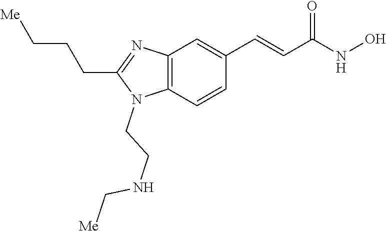 [SMILES: CCCCc1nc2cc(/C=C/C(=O)NO)ccc2n1CCNCC]

or a pharmaceutically acceptable salt, isotopic variant, or prodrug thereof.

In addition to compounds of Formula (I), the embodiments disclosed are also directed to pharmaceutically acceptable salts, pharmaceutically acceptable prodrugs, and isotopic variant of such compounds. Such compounds, salts, prodrugs and isotopic variant are at times collectively referred to herein as “HDAC inhibiting agents” or “HDAC inhibitors”.

Compounds of formula (I) described herein include the disclosure found in international application No.: PCT/SG2006/000217, entitled “HETEROCYCLIC COMPOUNDS”, filed on Aug. 1, 2006, which is incorporated herein in its entirety by reference. The compounds of formula (I) and the embodiments disclosed herein inhibit histone deacetylases. In certain embodiments, the histone deacetylase inhibitor interacts with and/or reduces the activity of more than one known histone deacetylase in the cell, which can either be from the same class of histone deacetylase or different class of histone deacetylase. In some other embodiments, the histone deacetylase inhibitor interacts and reduces the activity of predominantly one histone deacetylase, for example HDAC-1, HDAC-2, HDAC-3 or HDAC-8 which belongs to Class I HDAC enzymes. In some embodiments, the compounds of formula (I) have significant anti-proliferative effects and promote differentiation, cell cycle arrest in the G1 or G2 phase, and induce apoptosis.

### DNA Hypomethylating Agents

Any suitable hypomethylating agent may be used in combination with a compound of formula (I). DNA hypomethylating agents for use in the methods provided herein include but are not limited to 5-azacytidine (azacitidine), 5-azadeoxycytidine (decitabine), SGI-110, zebularine and procaine. In certain specific embodiments, the DNA hypomethylating agent is 5-azacytidine (azacitidine).

### Methods

Provided herein are methods of treating a disease or disorder associated with dysregulation of histone deacetylase, comprising administering to a patient or subject in need thereof an effective amount of (i) a DNA hypomethylating agent, and (ii) about 45 mg of a compound of formula (I) or a pharmaceutically acceptable salt, isotopic variant, or prodrug thereof. In some embodiments, the DNA hypomethylating agent acts additively with a compound of formula (I). In some embodiments, the DNA hypomethylating agent acts synergistically with a compound of formula (I). In some embodiments, a compound of formula (I) is pracinostat.

Some embodiments provided herein describe methods of treatment of a disorder caused by, associated with or accompanied by disruptions of cell proliferation and/or angiogenesis including administration of a therapeutically effective amount of a DNA hypomethylating agent and 45 mg of a compound of formula (I) or a pharmaceutically acceptable salt, isotopic variant, or prodrug thereof.

Also provided herein in some embodiments are agents for the treatment of a disorder caused by, associated with or accompanied by disruptions of cell proliferation and/or angiogenesis. In some embodiments, the agents are a DNA hypomethylating agent and a compound of formula (I) or a pharmaceutically acceptable salt, isotopic variant, or prodrug thereof.

Some embodiments described herein relate to the use of a DNA hypomethylating agent and a compound of formula (I) in the preparation of a medicament for the treatment of a disorder caused by, associated with or accompanied by disruptions of cell proliferation and/or angiogenesis. In one embodiment, the disorder is a proliferative disorder. In a specific embodiment, the disorder is a cancer. In some embodiments, the combination therapy of a DNA hypomethylating agent and a compound of formula (I) show low toxicity. In some embodiments, the combination therapy of a DNA hypomethylating agent and a compound of formula (I) show potent anti-proliferative activity.

Other embodiments described herein provide a method of treatment of a disorder, disease or condition that can be treated by the inhibition of histone deacetylase including administration of a therapeutically effective amount of a DNA hypomethylating agent and a compound of formula (I).

Also described herein are agents for the treatment of a disorder, disease or condition that can be treated by the inhibition of histone deacetylase. In one embodiment the agent is an anticancer agent. In some embodiments, the agents are a DNA hypomethylating agent and a compound of formula (I). Some embodiments described herein provide a method for inhibiting cell proliferation including administration of an effective amount of a DNA hypomethylating agent and a compound according to formula (I).

Provided herein in certain embodiments is a method of treating chemoresistant cancer comprising administering to a patient or subject in need thereof an effective amount of a DNA hypomethylating agent and a compound of formula (I). In some embodiments, the cancer is refractory, non-responsive or resistant to chemotherapy. In some embodiments, the cancer is refractory, non-responsive or resistant to haploidentical stem cell transplantation. In some embodiments, the cancer is resistant to azacitidine, decitabine, SGI-110, lenalidomide, TXA-127, or combinations thereof. In some embodiments, the cancer is resistant to azacitidine, decitabine, lenalidomide, TXA-127, or combinations thereof.

In some embodiments, the cancer is high or very high risk myelodysplastic syndrome (MDS) according to the IPSS-R. In some specific embodiments, the cancer is high risk myelodysplastic syndrome (MDS) according to the IPSS-R. In some specific embodiments, the cancer is very high risk myelodysplastic syndrome (MDS) according to the IPSS-R. In some embodiments, the MDS is refractory, non-responsive, or resistant to chemotherapy and/or haploidentical stem cell transplantation.

In some embodiments, the methods described herein are useful in treating various cancers including but not limited to bone cancers including Ewing's sarcoma, osteosarcoma, chondrosarcoma and the like, brain and CNS tumors including acoustic neuroma, neuroblastomas, glioma and other brain tumors, spinal cord tumors, breast cancers including ductal adenocarcinoma, metastatic ductal breast carcinoma, colorectal cancers, advanced colorectal adenocarcinomas, colon cancers, endocrine cancers including adrenocortical carcinoma, pancreatic cancer, pituitary cancer, thyroid cancer, parathyroid cancer, thymus cancer, multiple endocrine neoplasm, gastrointestinal cancers including stomach cancer, esophageal cancer, small intestine cancer, liver cancer, extra hepatic bile duct cancer, gastrointestinal carcinoid tumor, gall bladder cancer, genitourinary cancers including testicular cancer, penile cancer, prostate cancer, gynecological cancers including cervical cancer, ovarian cancer, vaginal cancer, uterus/endometrium cancer, vulva cancer, gestational trophoblastic cancer, fallopian tube cancer, uterine sarcoma, head and neck cancers including oral cavity cancer, lip cancer, salivary gland cancer, larynx cancer, hypopharynx cancer, oropharynx cancer, nasal cancer, paranasal cancer, nasopharynx cancer, leukemias including childhood leukemia, acute lymphocytic leukemia, acute myeloid leukemia, chronic lymphocytic leukemia, chronic myeloid leukemia, hairy cell leukemia, acute promyelocytic leukemia, plasma cell leukemia, erythroleukemia, myelomas, hematological disorders including myelodysplastic syndromes, myeloproliferative disorders, aplastic anemia, Fanconi anemia, Waldenstroms Macroglobulinemia, lung cancers including small cell lung cancer, non-small cell lung cancer, mesothelioma, lymphomas including Hodgkin's disease, non-Hodgkin's lymphoma, cutaneous T-cell lymphoma, peripheral T-cell lymphoma, AIDS related Lymphoma, B-cell lymphoma, Burkitt's lymphoma, eye cancers including retinoblastoma, intraocular melanoma, skin cancers including melanoma, non-melanoma skin cancer, squamous cell carcinoma, merkel cell cancer, soft tissue sarcomas such as childhood soft tissue sarcoma, adult soft tissue sarcoma, Kaposi's sarcoma, urinary system cancers including kidney cancer, Wilms tumor, bladder cancer, urethral cancer, and transitional cell cancer.

In some embodiments, the disease or disorder associated with dysregulation of histone deacetylase is cancer. In some embodiments, the cancer is a hematological malignancy. In some embodiments, wherein the hematological malignancy is acute myeloid leukemia (AML), chronic myeloid leukemia (CML), chronic myelomonocytic leukemia, thrombolytic leukemia, a myelodysplastic syndrome (MDS), a myeloproliferative disorder, refractory anemia, a preleukemia syndrome, a lymphoid leukemia, lymphoma, non-Hodgkin's lymphoma, or an undifferentiated leukemia. In some specific embodiments, the cancer is myelodysplastic syndrome (MDS) or acute myeloid leukemia (AML). Non-limiting examples of non-Hodgkin's lymphoma include diffuse large B-cell lymphoma (DLBCL), mantle cell lymphoma (MCL), and chronic lymphocytic leukemia (CLL).

Other exemplary cancers that may be treated by the methods described herein include but are not limited to leukemias such as erythroleukemia, acute promyelocytic leukemia, acute myeloid leukemia, acute lymphocytic leukemia, acute T-cell leukemia and lymphoma such as B-cell lymphoma (e.g. Burkitt's lymphoma), cutaneous T-cell lymphoma (CTCL), and peripheral T-cell lymphoma.

Certain exemplary cancers that may be treated by the methods described herein include solid tumors and hematologic malignancies. In another embodiment, preferred cancers that may be treated with the compounds of the present invention are colon cancer, prostate cancer, hepatoma and ovarian cancer.

In some embodiments, the patient in need thereof has not been previously treated with a DNA hypomethylating agent. In some embodiments, the patient in need thereof has not been previously treated with 5-azacitidine. In some embodiments, the patient in need thereof has not been previously treated with 5-azadeoxycytidine. In some embodiments, the patient in need thereof has been previously treated with transfusions, hematopoietic growth factors, or immunosuppressive therapy.

In some embodiments, the methods described herein provides a high overall response rate (ORR) as determined by tumor assessment from radiological tests and/or physical examination. In some instances, response evaluation is performed after 2 and 6 cycles of therapy, and then every 6 months or as clinically indicated. In some embodiments, the methods described herein provide complete remission. In some embodiments, the methods described herein provide complete remission beginning within 12 months of treatment and lasting ≥6 months. In some embodiments, the methods described herein provide a complete response (CR) and/or no evidence of disease (NED) beginning within 12 months of treatment and lasting ≥6 months. In some embodiments, the overall response rate evaluated after 6 cycles of therapy is ≥20%. In some embodiments, the overall response rate evaluated after 6 cycles of therapy is ≥25%. In some embodiments, the overall response rate evaluated after 6 cycles of therapy is ≥30%. In some embodiments, the overall response rate evaluated after 6 cycles of therapy is ≥35%. In some embodiments, the overall response rate evaluated after 6 cycles of therapy is ≥40%.

In some embodiments, the methods of treatment and dosing schedules described herein improve the frequency, severity and time to onset of adverse events (AEs) in patients receiving the treatment described herein. In some embodiments, the adverse event is selected from constipation, nausea, fatigue, decreased appetite, diarrhea, edema peripheral, hypoalbuminemia, dyspnea, hypokalemia, vomiting, dizziness, febrile neutropenia, anemia, neutropenia, and thrombocytopenia.

In some embodiments, the methods of treatment and dosing schedules described herein avoid or reduce adverse or unwanted side effects associated with the use of the HDAC inhibitors, such as constipation, nausea, fatigue, decreased appetite, diarrhea, edema peripheral, hypoalbuminemia, dyspnea, hypokalemia, vomiting, dizziness, febrile neutropenia, anemia, neutropenia, and thrombocytopenia. In some embodiments, the methods of treatment and dosing schedules described herein avoid or reduce cytopenia, nausea, vomiting, fatigue, or combinations thereof in patients receiving the treatment described herein. In certain embodiments, the methods described herein avoid, reduce, or minimize the incidence of cytopenia. In certain embodiments, the methods described herein avoid, reduce, or minimize the incidence of nausea. In certain embodiments, the methods described herein avoid, reduce, or minimize the incidence of vomiting. In certain embodiments, the methods described herein avoid, reduce, or minimize the incidence of fatigue.

In some embodiments, the discontinuation rate due to adverse events is less than 25%, less than 20%, less than 15%, less than 10%, less than 8%, less than 5%. In some embodiments, the discontinuation rate due to adverse events is less than 25%. In some embodiments, the discontinuation rate due to adverse events is less than 20%. In some embodiments, the discontinuation rate due to adverse events is less than 15%. In some embodiments, the discontinuation rate due to adverse events is less than 10%. In some embodiments, the discontinuation rate due to adverse events is less than 8%. In some embodiments, the discontinuation rate due to adverse events is about 4%.

In some embodiments, the discontinuation rate due to adverse events is less than the discontinuation rate observed when the patients are administered about 60 mg of a compound of formula (I) and a DNA hypomethylating agent.

In some embodiments, the discontinuation rate due to adverse events is less than the discontinuation rate observed when the patients are administered a DNA hypomethylating agent alone.

### Doses

The amount of compound of formula (I) administered to a patient in need thereof will be dependent on the patient or subject treated. In some embodiments, the amount of a compound of formula (I) administered is less than about 60 mg. In some embodiments, the amount of a compound of formula (I) administered is between about 10 mg and about 55 mg. In some embodiments, the amount of a compound of formula (I) administered is between about 20 mg and about 55 mg. In some embodiments, the amount of a compound of formula (I) administered is between about 30 mg and about 55 mg. In some embodiments, the amount of a compound of formula (I) administered is between about 40 mg and about 55 mg. In some embodiments, the amount of a compound of formula (I) administered is about 10 mg, about 15 mg, about 20 mg, about 25 mg, about 30 mg, about 35 mg, about 40 mg, about 45 mg, about 50 mg, or about 55 mg. In some embodiments, the amount of a compound of formula (I) administered is about 45 mg. In certain embodiments, about 45 mg of pracinostat is administered.

The amount of a DNA hypomethylating agent will be dependent on the patient or subject treated. In some instances where the patient or subject is a human, the dose will normally be determined by the prescribing physician with the dosage generally varying according to the age, sex, diet, weight, general health and response of the individual patient, the severity of the patient's symptoms, the precise indication or condition being treated, the severity of the indication or condition being treated, time of administration, route of administration, the disposition of the composition, rate of excretion, and the discretion of the prescribing physician. In some embodiments, the total dosage for the day is divided and administered in portions during the day if desired. In some embodiments, combinational applications in which the combination therapy described herein is not the sole therapy, allows for the administration of lesser amounts of a DNA hypomethylating agent and a compound of formula (I).

In specific embodiments, an effective amount of DNA hypomethylating agent is from about 5 mg/m² to about 1000 mg/m², from about 5 mg/m² to about 125 mg/m², from about 10 mg/m² to about 1000 mg/m², from about 10 mg/m² to about 800 mg/m², from about 10 mg/m² to about 700 mg/m², from about 10 mg/m² to about 600 mg/m², from about 10 mg/m² to about 500 mg/m², from about 10 mg/m² to about 400 mg/m², from about 10 mg/m² to about 350 mg/m², from about 10 mg/m² to about 300 mg/m², from about 10 mg/m² to about 200 mg/m², from about 10 mg/m² to about 175 mg/m², from about 10 mg/m² to about 150 mg/m², from about 10 mg/m² to about 125 mg/m², from about 10 mg/m² to about 115 mg/m², from about 10 mg/m² to about 100 mg/m², from about 10 mg/m² to about 80 mg/m², from about 10 mg/m² to about 60 mg/m², from about 10 mg/m² to about 20 mg/m², from about 5 mg/m² to about 20 mg/m², from about 50 mg/m² to about 500 mg/m², from about 50 mg/m² to about 400 mg/m², from about 10 mg/m² to about 350 mg/m², from about 50 mg/m² to about 300 mg/m², from about 50 mg/m² to about 250 mg/m², from about 50 mg/m² to about 225 mg/m², from about 50 mg/m² to about 200 mg/m², from about 50 mg/m² to about 175 mg/m², from about 50 mg/m² to about 150 mg/m², from about 50 mg/m² to about 125 mg/m², from about 50 mg/m² to about 100 mg/m², from about 50 mg/m² to about 90 mg/m², from about 50 mg/m² to about 80 mg/m², from about 60 mg/m² to about 80 mg/m², from about 75 mg/m² to about 250 mg/m², from about 75 mg/m² to about 200 mg/m², from about 75 mg/m² to about 150 mg/m², from about 75 mg/m² to about 125 mg/m², less than 1000 mg/m², less than 900 mg/m², less than 800 mg/m², less than 700 mg/m², less than 600 mg/m², less than 500 mg/m², less than 400 mg/m², less than 350 mg/m², less than 300 mg/m², less than 275 mg/m², less than 250 mg/m², less than 225 mg/m², less than 200 mg/m², less than 175 mg/m², less than 150 mg/m², less than 125 mg/m², less than 115 mg/m², less than 100 mg/m², less than 90 mg/m², less than 80 mg/m², less than 70 mg/m², less than 60 mg/m², less than 50 mg/m², less than 40 mg/m², less than 30 mg/m², less than 20 mg/m², less than 10 mg/m², about 1000 mg/m², about 900 mg/m², about 800 mg/m², about 700 mg/m², about 600 mg/m², about 500 mg/m², about 400 mg/m², about 350 mg/m², about 300 mg/m², about 250 mg/m², about 225 mg/m², about 200 mg/m², about 175 mg/m², about 150 mg/m², about 140 mg/m², about 130 mg/m², about 125 mg/m², about 115 mg/m², about 100 mg/m², about 90 mg/m², about 95 mg/m², about 90 mg/m², about 85 mg/m², about 80 mg/m², about 75 mg/m², about 70 mg/m², about 65 mg/m², about 60 mg/m², about 50 mg/m², about 55 mg/m², about 45 mg/m², about 40 mg/m², about 35 mg/m², about 30 mg/m², about 25 mg/m², about 20 mg/m², about 15 mg/m², about 11 mg/m², about 10 mg/m², about 5 mg/m², or about 2 mg/m². In certain specific embodiment, an effective amount of a DNA hypomethylating agent administered according to any of the methods described herein is about 75 mg/m². In certain embodiments, about 75 mg/m² of 5-azacitidine is administered.

### Administration

In some embodiments, a compound of formula (I) and a DNA hypomethylating agent are administered to a patient or subject (e.g., a human) by any acceptable modes for enteral administration such as oral or rectal, or by parenteral administration such as subcutaneous, intramuscular, intravenous and intradermal routes. In some embodiments, injection is bolus or via constant or intermittent infusion. In various embodiments, the combination of a compound of formula (I) and a DNA hypomethylating agent is selectively toxic or more toxic to rapidly proliferating cells, e.g. cancerous tumors, than to normal cells.

The compounds of the present invention can be administered alone or in the form of a pharmaceutical composition in combination with a pharmaceutically acceptable carrier, diluent or excipient. The compounds of the invention, while effective themselves, are typically formulated and administered in the form of their pharmaceutically acceptable salts as these forms are typically more stable, more easily crystallized and have increased solubility.

In some embodiments, a compound of formula (I) and a DNA hypomethylating agent are used in the form of pharmaceutical compositions which are formulated depending on the desired mode of administration. In some embodiments, a pharmaceutical composition includes a compound of formula (I) and a pharmaceutically acceptable carrier, diluent or excipient. In other embodiments, a pharmaceutical composition includes a DNA hypomethylating agent and a pharmaceutically acceptable carrier, diluent or excipient. In certain embodiments, a pharmaceutical composition includes a compound of formula (I), a DNA hypomethylating agent, and at least one pharmaceutically acceptable carrier, diluents or excipient. In certain specific embodiments, the compound of formula (I) is pracinostat. In certain specific embodiments, the DNA hypomethylating agent is 5-azacitidine. In certain specific embodiments, the DNA hypomethylating agent is 5-azadeoxycytidine.

Some embodiments provided herein describe pharmaceutical compositions for parenteral injection comprising pharmaceutically acceptable sterile aqueous or nonaqueous solutions, dispersions, suspensions or emulsions as well as sterile powders for reconstitution into sterile injectable solutions or dispersions just prior to use. Non-limiting examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. In some embodiments, proper fluidity is maintained, for example, by the use of coating materials such as lecithin, by the maintenance of the required particle size in the case of dispersions, and by the use of surfactants.

In some embodiments, provided herein are compositions containing adjuvants such as preservative, wetting agents, emulsifying agents, and dispersing agents. In some embodiments, various antibacterial and antifungal agents, for example, paraben, chlorobutanol, phenol sorbic acid, and the like are included to prevent the action of microorganisms. In some embodiments, the pharmaceutical composition includes isotonic agents such as sugars, sodium chloride, and the like. In some embodiments, prolonged absorption of the injectable pharmaceutical form is brought about by the inclusion of agents that delay absorption such as aluminum monostearate and gelatin.

In some embodiments, for more effective distribution, the active agents are incorporated into slow release or targeted delivery systems such as polymer matrices, liposomes, and microspheres.

In certain embodiments, the injectable formulation is sterilized, for example, by filtration through a bacterial-retaining filter, or by incorporating sterilizing agents in the form of sterile solid compositions that are dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

In some embodiments, solid compositions comprise fillers in soft and hard-filled gelatin capsules using such excipients as lactose or milk sugar as well as high molecular weight polyethylene glycols and the like.

In some instances, the solid dosage forms of tablets, dragees, capsules, pills, and granules are prepared with coatings and shells such as enteric coatings and other coatings well known in the pharmaceutical formulating art. In some embodiments, the solid dosage forms optionally contain opacifying agents and release the active ingredient(s) only, or preferentially, in a certain part of the intestinal tract, optionally, in a delayed manner. Examples of embedding compositions which can be used include polymeric substances and waxes.

In some embodiments, the compounds are incorporated into slow release or targeted delivery systems such as polymer matrices, liposomes, and microspheres.

In some instances, the active compounds are in microencapsulated form, if appropriate, with one or more of the above-mentioned excipients.

Liquid dosage forms for oral administration include pharmaceutically acceptable emulsions, solutions, suspensions, syrups and elixirs. In some instances, the liquid dosage forms contains inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

In some instances, the oral compositions also include adjuvants such as wetting agents, emulsifying and suspending agents, sweetening, flavoring, and perfuming agents.

In some embodiments, any compound of formula (I) is administered intravenously, subcutaneously, or orally. In certain embodiments, a compound of formula (I) is administered orally. In certain specific embodiments, pracinostat is administered orally. In certain specific embodiments, pracinostat is administered intravenously.

In some embodiments, a DNA hypomethylating agent is administered intravenously, subcutaneously, or orally. In certain embodiments, a DNA hypomethylating agent is administered intravenously. In other embodiments, a DNA hypomethylating agent is administered subcutaneously. In certain specific embodiments, 5-azacitidine is administered intravenously. In other specific embodiments, 5-azacitidine is administered subcutaneously. In certain specific embodiments, 5-azadeoxycytidine is administered intravenously. In other specific embodiments, 5-azadeoxycytidine is administered subcutaneously.

Some embodiments provided herein describe a combination therapy comprising a compound of formula (I) and a DNA hypomethylating agent, wherein the compound of formula (I) and the DNA hypomethylating agent are administered in combination with each other. In some instances, the compound of formula (I) and the DNA hypomethylating agent are administered simultaneously. In other instances, the compound of formula (I) and the DNA hypomethylating agent are administered sequentially. In other instances, the compound of formula (I) and the DNA hypomethylating agent are administered within the same week.

In some embodiments, the compound of formula (I) is administered daily, every other day, every other day 3 times a week, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the DNA hypomethylating agent is administered daily, every other day, every other day 3 times a week, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months.

In some instances, the compound of formula (I) or the DNA hypomethylating agent is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 8 days, 9 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In some embodiments, the compound of formula (I) and the DNA hypomethylating agent are administered in multiple chemotherapy cycles. In some embodiments, the compound of formula (I) and the DNA hypomethylating agent are administered in 28 days cycles. In some embodiments, the compound of formula (I) and the DNA hypomethylating agent are administered for at least 3 cycles. In some embodiments, the compound of formula (I) and the DNA hypomethylating agent are administered for at least 4 cycles. In some embodiments, the compound of formula (I) and the DNA hypomethylating agent are administered for at least 5 cycles. In some embodiments, the compound of formula (I) and the DNA hypomethylating agent are administered for at least 6 cycles. In some embodiments, the compound of formula (I) and the DNA hypomethylating agent are administered for at least 7 cycles. In some embodiments, the compound of formula (I) and the DNA hypomethylating agent are administered for at least 8 cycles. In some embodiments, the compound of formula (I) and the DNA hypomethylating agent are administered for at least 9 cycles. In some embodiments, the compound of formula (I) and the DNA hypomethylating agent are administered for at least 10 cycles.

In some embodiments, the compound of formula (I) and the DNA hypomethylating agent are administered until complete remission (CR) is observed.

In some instances, the compound of formula (I) is administered orally once per day, 3 days each week for 3 weeks, followed by 1 week of rest. Treatment cycles are repeated every 28 days, unless delayed due to toxicity.

In some embodiments, the dosing interval between two consecutive doses of the compound of formula (I) is about 48 hours.

In some embodiments, in later cycles (e.g., after Cycle 4), 45 mg of the compound of formula (I) is orally administered to the patient 3 days each week for 2 weeks of each 28-day cycle. In some embodiments, dose interruption is allowed to manage toxicity such as fatigue, GI toxicity, or myelosuppression.

In some embodiments, the methods described herein comprise administering to the patient a DNA hypomethylating agent at 75 mg/m² for 7 days of each 28-day cycle. In some embodiments, the administration occurs by SC injection or IV infusion if SC injections are not tolerated, on one of two schedules:

Schedule 1— daily therapy on Days 1 through 7; or
Schedule 2—5-2-2 schedule in which the DNA hypomethylating agent is administered to the patient for 5 consecutive days (Days 1 through 5) with rest on Days 6 and 7, and resume the DNA hypomethylating agent dosing the first two days of the next week (Days 8 and 9) of each 28-day cycle.

In certain embodiments, the method of treating high or very high risk myelodysplastic syndromes (MDS) in a patient in need thereof comprises administering to the patient (i) about 75 mg/m² of 5-azacytidine (azacitidine), wherein 5-azacytidine (azacitidine) is administered intravenously or subcutaneously for 7 days of each 28-day cycle; and (ii) about 45 mg of pracinostat, or a pharmaceutically acceptable salt, isotopic variant, or prodrug thereof, wherein pracinostat is orally administered for 3 days each week for 3 consecutive weeks, followed by 1 week of rest, in 28-day cycles. In further or additional embodiments, the combination therapy (5-azacytidine (azacitidine) and pracinostat or a pharmaceutically acceptable salt, isotopic variant, or prodrug thereof) are administered for at least 3 cycles. In further or additional embodiments, the combination therapy (5-azacytidine (azacitidine) and pracinostat or a pharmaceutically acceptable salt, isotopic variant, or prodrug thereof) are administered for at least 4 cycles. In further or additional embodiments, the combination therapy (5-azacytidine (azacitidine) and pracinostat or a pharmaceutically acceptable salt, isotopic variant, or prodrug thereof) are administered for at least 5 cycles. In further or additional embodiments, the combination therapy (5-azacytidine (azacitidine) and pracinostat or a pharmaceutically acceptable salt, isotopic variant, or prodrug thereof) are administered for at least 6 cycles. In further or additional embodiments, the combination therapy (5-azacytidine (azacitidine) and pracinostat or a pharmaceutically acceptable salt, isotopic variant, or prodrug thereof) are administered for at least 3 cycles, followed by further administration of pracinostat, or a pharmaceutically acceptable salt, isotopic variant, or prodrug thereof, for 3 days each week for 2 consecutive weeks, followed by 2 weeks of rest for at least one 28-day cycle.

In certain embodiments, the method of treating very high risk myelodysplastic syndromes (MDS) in a patient in need thereof comprises administering to the patient (i) about 75 mg/m² of 5-azacytidine (azacitidine), wherein 5-azacytidine (azacitidine) is administered intravenously or subcutaneously for 7 days of each 28-day cycle; and (ii) about 45 mg of pracinostat, or a pharmaceutically acceptable salt, isotopic variant, or prodrug thereof, wherein pracinostat is orally administered for 3 days each week for 3 consecutive weeks, followed by 1 week of rest, in 28-day cycles. In further or additional embodiments, the combination therapy (5-azacytidine (azacitidine) and pracinostat or a pharmaceutically acceptable salt, isotopic variant, or prodrug thereof) are administered for at least 3 cycles. In further or additional embodiments, the combination therapy (5-azacytidine (azacitidine) and pracinostat or a pharmaceutically acceptable salt, isotopic variant, or prodrug thereof) are administered for at least 4 cycles. In further or additional embodiments, the combination therapy (5-azacytidine (azacitidine) and pracinostat or a pharmaceutically acceptable salt, isotopic variant, or prodrug thereof) are administered for at least 5 cycles. In further or additional embodiments, the combination therapy (5-azacytidine (azacitidine) and pracinostat or a pharmaceutically acceptable salt, isotopic variant, or prodrug thereof) are administered for at least 6 cycles. In further or additional embodiments, the combination therapy (5-azacytidine (azacitidine) and pracinostat or a pharmaceutically acceptable salt, isotopic variant, or prodrug thereof) are administered for at least 3 cycles, followed by further administration of pracinostat, or a pharmaceutically acceptable salt, isotopic variant, or prodrug thereof, for 3 days each week for 2 consecutive weeks, followed by 2 weeks of rest for at least one 28-day cycle.

Some embodiments provided herein describe a combination therapy that is used or administered in combination with one or more additional drug (s) that are chemotherapeutic drugs or HDAC inhibitor drugs and/or procedures (e.g. surgery, radiotherapy) for the treatment of the disorder/diseases mentioned. In some embodiments, the additional drug(s) are administered in the same formulation or in separate formulations. In some embodiments, if administered in separate formulations, the combination therapy is administered sequentially or simultaneously (as a combined preparation) with the additional drug(s).

### Kits

Some embodiments provided herein describe a pharmaceutical pack or kit comprising one or more containers filled with a compound of formula (I) and one or more containers filled with a DNA hypomethylating agent. In some embodiments, the kit comprises one container filled with a compound of formula (I) and a DNA hypomethylating agent. In some embodiments, the kit comprises a container having a unit dosage of the agent(s). In certain embodiments, the kits include one or more compositions comprising a compound of formula (I) and a DNA hypomethylating agent (including lyophilized compositions), which can be diluted further prior to use or they can be provided at the concentration of use, where the vials may include one or more dosages. Conveniently, in the kits, single dosages can be provided in sterile vials so that the physician can employ the vials directly, where the vials will have the desired amount and concentration of agent(s). Associated with such container(s) can be various written materials such as instructions for use, or a notice in the form prescribed by a governmental agency regulating the manufacture, use or sale of pharmaceuticals or biological products, which notice reflects approval by the agency of manufacture, use or sale for human administration.

In some embodiments, the kit comprises a container filled with 3-[2-butyl-1-(2-diethylamino-ethyl)-1H-benzimidazol-5-yl]-N-hydroxy-acrylamide and 5-azacitidine. In other embodiments, the kit comprises a container filled with 3-[2-butyl-1-(2-diethylamino-ethyl)-1H-benzimidazol-5-yl]-N-hydroxy-acrylamide and 5-azadeoxycytidine. In some embodiments, the kit comprises one or more containers filled with 3-[2-butyl-1-(2-diethylamino-ethyl)-1H-benzimidazol-5-yl]-N-hydroxy-acrylamide and one or more containers filled with 5-azacitidine. In other embodiments, the kit comprises one or more containers filled with 3-[2-butyl-1-(2-diethylamino-ethyl)-1H-benzimidazol-5-yl]-N-hydroxy-acrylamide and one or more containers filled with 5-azadeoxycytidine.

### EXAMPLES

### Example 1: Human Clinical Trial with Pracinostat and Azacitidine in Patients with IPSS-R High and Very High Risk Myelodysplastic Syndromes Previously Untreated with Hypomethylating Agents

### Study Design

This is a multicenter, open-label, two-stage design, Phase 2 study being conducted at 24 sites.

Up to 40 subjects were to enroll in Stage 1, and those enrolled were treated with pracinostat at 45 mg, 3 days each week for 3 consecutive weeks, followed by 1 week of rest, along with AZA at a dose of 75 mg/m² for 7 days of each 28-day cycle.

Study drugs were administered until disease progression, intolerable toxicity, or referral for allogeneic stem cell transplantation (SCT), avoiding early discontinuation (<6 months) due to lack of response.

### Key Inclusion Criteria

Age≥65 years with histologically or cytologically documented MDS according to WHO classification
Classified as high- or very high-risk MDS according to the IPSS-R
CMML-1 and CMML-2 subtypes were allowed
Bone marrow biopsy and/or aspirate within 28 days prior to Day 1
Eastern Cooperative Oncology Group (ECOG) performance score of 0-2
Previously untreated with HMAs (prior therapy with transfusions, hematopoietic growth factors, or immunosuppressive therapy is allowed) and clinical indication for treatment with AZA

### Key Exclusion Criteria

Bone marrow blasts≥20%, indicating a diagnosis of acute myeloid leukemia (AML)
Received any investigational agent within 14 days or 5 half-lives prior to enrollment, hydroxyurea within 48 hours prior to first day of study treatment, or hematopoietic growth factors: erythropoietin, granulocyte colony stimulating factor (G-CSF), granulocyte macrophage colony stimulating factor (GM-CSF), or thrombopoietin receptor agonists at least 7 days (14 days for Aranesp) prior to study enrollment
Major surgery within 28 days prior to first study treatment
Current unstable arrhythmia requiring treatment, history of symptomatic congestive heart failure (New York Heart Association [NYHA] Class III or IV), history of myocardial infarction within 6 months of enrollment, or current unstable angina
Prior treatment for MDS with the HDAC inhibitor or investigational agent with significant action as an HDAC inhibitor

### Results

The interim analysis for expansion was performed in May 2018; 40 patients have been enrolled and received ≥1 dose of study drug, of which 20 patients were deemed evaluable, having received ≥3 cycles of therapy or having discontinued study drug due to an adverse event prior to start of Cycle 4.

2/20 (10%) discontinued due to adverse events in the first 3 cycles, 1 after 1 cycle due neutropenic fever and the other after 2 cycles due to a fungal esophagitis and neutropenic fever.

In 18 subjects evaluable for response assessment at the end of Cycle≥2, the ORR was 28% (1 complete response, 4 partial responses).

This early discontinuation rate of 10% met the preplanned interim analysis threshold for expansion and the IDMC authorized expansion to Stage 2.

### Baseline Characteristics

Between June 2017 and October 2018, a total of 55 patients were enrolled at 17 US centers and received ≥1 dose of study treatment. The majority of patients were male (64%) with a median age of 68 years. The median time from diagnosis was 1 month and patients were generally split between high- and very high-risk MDS.

### Patient Disposition

As of Oct. 25, 2018, 23 patients (42%) had discontinued study drugs (Table 1)

Five patients (9%) discontinued due to adverse events, of which 2 (4%) were considered early discontinuations (within the first 3 months/cycles).

For the 32 ongoing patients (58%), the median duration on therapy is 4.7 months (range, 0.5-13 months). 14 patients (25%) had received >6 cycles of therapy.

TABLE 1
Reasons for Discontinuation (N = 23)

| | No. of | |
|---|---|---|
| | Patients (%) | |
| Reason | N = 55 | Week of Discontinuation |
| Allogeneic stem cell | 8 (15%) | 12, 16, 19, 25, 30, 32, 34, 39 |
| transplant (SCT) | | |
| Withdrawal of Consent | 6 (11%) | 0*, 9, 16, 20, 21, 40 |
| Adverse Event | 5 (9%) | 0*, 7, 13, 15, 39 |
| Progressive Disease | 3 (5%) | 6, 8, 34 |
| Physician | 1 (2%) | 0* |
| Decision/Insurance | | |

*discontinued on Day 1 after 1 dose of study drug;

### Safety

The most frequent non-hematologic AEs were constipation (490%), nausea (47%), fatigue (35%) and decreased appetite (35%); the most frequent hematologic AEs were febrile neutropenia (40%) and anemia (35%) (Table 2).

TABLE 2
Adverse Events in ≥20% of Patients (N = 55)

| | | |
|---|---|---|
| | Grade ≥3 | All Grades |
| Adverse Event | n (%) | n (%) |

Non-Hematologic

| | | | |
|---|---|---|---|
| Constipation | 0 | (0) | 27 (49) |
| Nausea | 1 | (2) | 26 (47) |
| Fatigue | 4 | (7) | 19 (35) |
| Decreased appetite | 1 | (2) | 19 (35) |
| Diarrhea | 1 | (2) | 15 (27) |
| Edema peripheral | 0 | (0) | 14 (26) |
| Hypoalbuminemia | 2 | (4) | 13 (24) |
| Dyspnea | 2 | (4) | 12 (22) |
| Hypokalemia | 1 | (2) | 12 (22) |
| Vomiting | 2 | (4) | 11 (20) |
| Dizziness | 0 | (0) | 11 (20) |

Hematologic

| | | | |
|---|---|---|---|
| Febrile neutropenia | 21 | (38) | 22 (40) |
| Anemia | 18 | (33) | 19 (35) |
| Neutropenia | 18 | (33) | 18 (33) |
| Thrombocytopenia | 14 | (26) | 15 (27) |

The incidences of key adverse events that led to early discontinuations in the prior study were lower in the current study for most of the AEs (Table 3).

TABLE 3
Key Adverse Events in Current Study Compared to
Prior Study (Garcia-Manero G, et al. Cancer 2017)

| | Grade ≥3 (% Patients) | | All Grades (% Patients) | |
|---|---|---|---|---|
| | Current | Prior | Current | Prior |
| | Study | Study | Study | Study |
| Adverse Event | N = 55 | N = 51 | N = 55 | N = 51 |

Non-Hematologic

| | | | | |
|---|---|---|---|---|
| Constipation | 0 | 2 | 49 | 53 |
| Nausea | 2 | 4 | 47 | 69 |
| Fatigue | 7 | 24 | 35 | 55 |
| Vomiting | 4 | 4 | 20 | 47 |

Hematologic

| | | | | |
|---|---|---|---|---|
| Febrile | 38 | 33 | 40 | 33 |
| neutropenia | | | | |
| Anemia | 33 | 20 | 35 | 31 |
| Neutropenia | 33 | 45 | 33 | 45 |
| Thrombocytopenia | 26 | 47 | 27 | 49 |

### Deaths

Nine patients (16% o) have died, 5 due to PD (progressive disease) and 4 due to AEs (adverse events); of these 5 patients (9% o) died while on study or within 28 days of discontinuing study drug (2 due to PD, 3 due to AEs).

Two patients died in the first 2 months on study, for a 60-day all-cause mortality of 3.6% o.

### Efficacy

As of Oct. 25, 2018, in the 45 patients deemed evaluable for response, the ORR (overall response rate) was 29% o (Table 4).

TABLE 4
Best Disease Response

| | | All Patients | | | |
|---|---|---|---|---|---|
| | Patients | Evaluable | | All Patients | |
| | Evaluable | for Response | | Who Reached | |
| | for Efficacy | at Cycle 2 | | Cycle 6 or | |
| | from the Interim | and/or at | | Discontinued | |
| | Analysis Subset* | Later Cycles* | | Before Cycle | |
| Endpoint | (N = 19) | (N = 45) | | 6** (N = 33) | |
| Complete | 7 (37%) | 13 | (29%) | 12 | (36%) |
| response (CR) | | | | | |
| Partial | — | — | | — | |
| Response (PR) | | | | | |
| Overall Response | 7 (37%) | 13 | (29%) | 12 | (36%) |
| Rate (ORR) | | | | | |
| Marrow CR + | 2 (10%) | 5 | (11%) | 4 | (12%) |
| Hematologic | | | | | |
| Improvement (HI) | | | | | |
| Marrow CR | 2 (10%) | 9 | (20%) | 6 | (18%) |
| Stable | 3 (16%) | 4 | (9%) | 4 | (12%) |
| Disease + HI | | | | | |
| Stable Disease | 3 (16%) | 11 | (24%) | 4 | (12%) |
| Progressive | 2 (10%) | 3 | (7%) | 3 | (9%) |
| Disease (PD) | | | | | |

*At least one disease assessment after baseline
**Hematologic improvement in at least 1 cell lineage

This study evaluating the efficacy and safety of pracinostat+AZA in patients with high-/very high-risk MDS showed that the lower dose of 45 mg pracinostat is better tolerated than the 60 mg dose evaluated in the prior study.

The incidences of adverse events that led to early discontinuations in the prior study were lower for non-haematological events and at least comparable for haematological events in the current study; it is noteworthy that patients in this study were higher-risk MDS than the prior study.

A discontinuation rate due to adverse events in the first 3 months of 4% is substantially lower than the rate of 26% reported in the prior study (Garcia-Manero G, et al. Cancer 2017) and to that reported in prior studies with AZA alone.

The complete remission rate was 29%.

An interim analysis was performed on Jul. 1, 2019. 64 patients have been enrolled and received ≥1 dose of study drug.

| | N = 64 | |
|---|---|---|
| MDS type by WHO classification | | |
| MDS EB-1 | 16 | (25%) |
| MDS-EB2 | 29 | (45%) |
| MDS-Multilineage dysplasia | 14 | (22%) |
| MDS ringed sideroblasts | 1 | (2%) |
| MDS with isolated del (5q) | 1 | (2%) |
| MDS unclassifiable with single lineage dysplasia | 1 | (2%) |
| Not available | 2 | (3%) |
| IPSS-R score | | |
| Very high risk | 30 | (47%) |
| High risk | 32 | (50%) |
| Intermediate risk | 1 | (1.5%) |
| Not available | 1 | (1.5%) |
| CMML | 4 | (6%) |

MDS = myelosysplastic syndrome
EB = Excess blasts
Del(5q) = Deletion chromosome 5q
IPSS-R = International Prognostic Scoring Sysntem-Revised
CMML = Chronic myelomonocytic leukemia

### Patient Disposition

As of Jul. 1, 2019, 39 patients (61%) had discontinued study drugs (Table 5)

TABLE 5
Reasons for Discontinuation (N = 39)

| | No. of Patients (61%) |
|---|---|
| Reason | N = 64 |
| Allogeneic stem cell transplant (SCT) | 15 |
| Adverse Event | 4 |
| Progressive Disease | 5 |
| Other: | 15 |
| Withdrawal of consent | 4 |
| Other (1 death due to AE) | 6 |
| Death (both due to EA) | 2 |
| Non-compliance by subject | 2 |
| Lost to follow-up | 1 |

AE = Adverse event

### Efficacy

The best response rates as classified by the International Working Group (IWG) criteria is shown in Table 6 and the hematologic improvement by cell lineage is shown in Table 7.

TABLE 6
Best Disease Response

| | Patients Evaluable for Efficacy |
|---|---|
| Endpoint | from the Interim Analysis (N = 60*) |
| Complete response (CR) | 20 (33%) |
| Partial Response (PR) | 1 (2%) |
| Marrow CR (mCR) | 23 (38%) |
| Stable Disease | 13 (22%) |
| Progressive Disease (PD) | 3 (5%) |

*4 patients did not have post-baseline follow-up bone marrow assessment and are not evaluable.

TABLE 7
Hematologic improvement by cell lineage

| Cell lineage | HI Rate* |
|---|---|
| Erythroid | 33/56 (59%) |
| Platelets | 35/51 (69%) |
| Neutrophils | 24/35 (69%) |

*Number of patients with hematologic improvement/Number evaluable patients

### Example 2: Comparison to Other AZA Studies

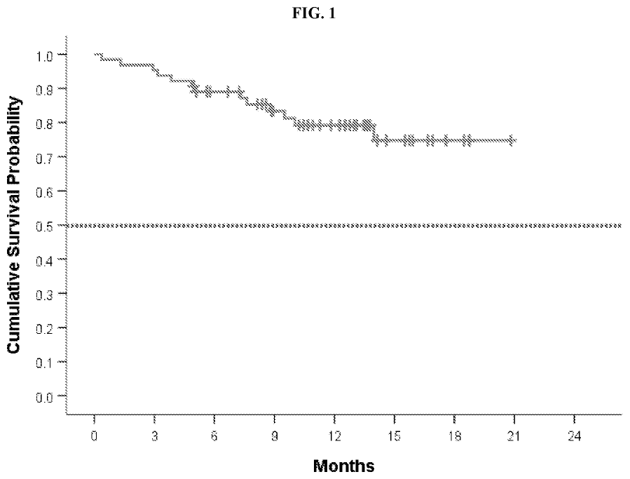

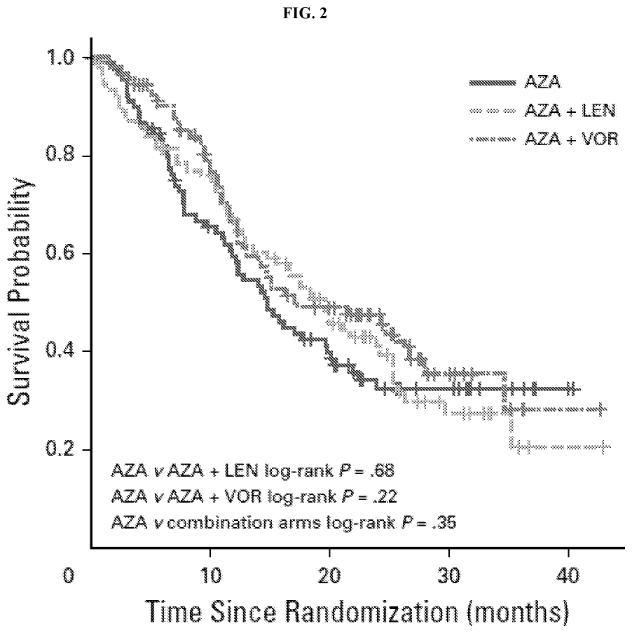

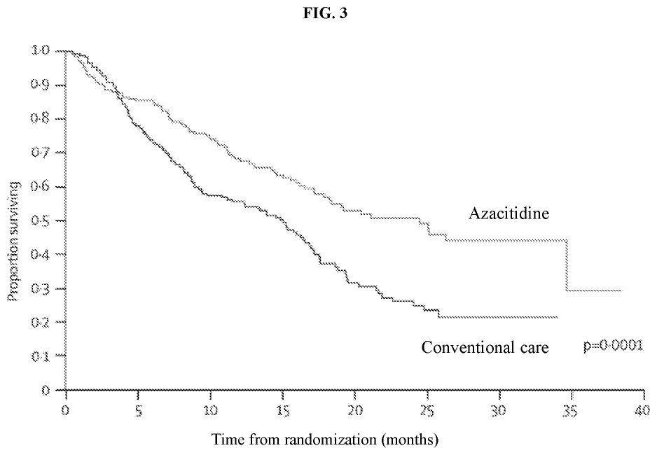

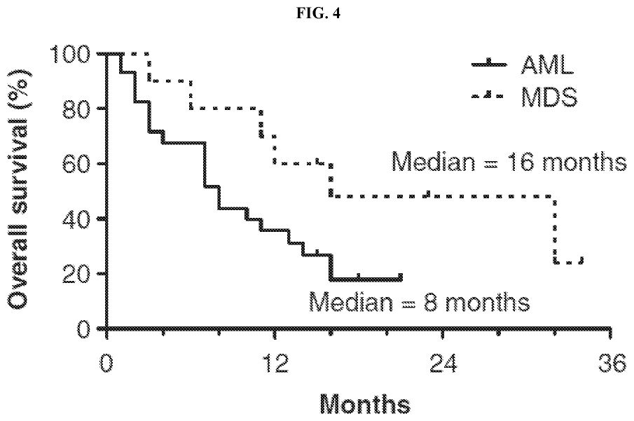

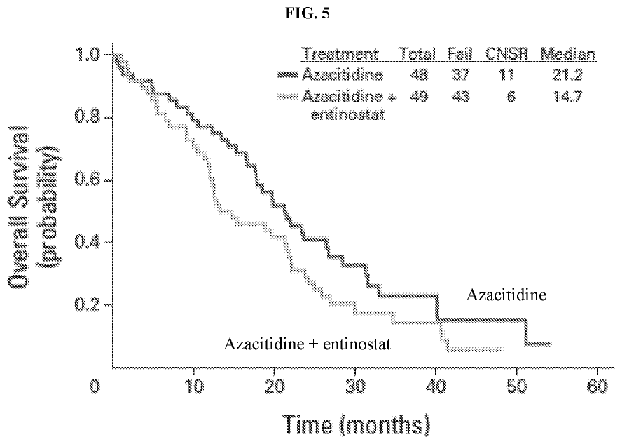

The overall survival following treatment with pracinostat+azacitidine (FIG. 1) was compared to other treatments as can be seen in study #1 (and FIG. 2), study #2 (and FIG. 3), study #3 (and FIG. 4), and study #4 (and FIG. 5).

Study #1: Randomized Phase II Study of Azacitidine Alone or in Combination With Lenalidomide or With Vorinostat in Higher-Risk Myelodysplastic Syndromes and Chronic Myelomonocytic Leukemia: North American Intergroup Study SWOG S 1117 (Sekeres et al, JCO 2017)

| | AZA | AZA + | AZA + | Total | PRAC-AZA |
|---|---|---|---|---|---|
| | Monotherapy | Lenalidomide | Vorinostat | 3 arms | Evaluable |
| | (N = 92) | (N = 93) | (N = 92) | (N = 277) | (N = 60) |
| ORR = CR + | 38 | 49 | 27 | 38 | 73 |
| PR + mCR (%) | | | | | |
| CR/PR/HI (%) | 24/0/14 | 24/1/25 | 17/1/9 | 22/1/16 | 31/2/36 |
| Marrow CR (%) | 12 | 9 | 14 | 12 | 38 |
| Allogeneic SCT (%) | 16 | 11 | 16 | 14 | 25 |

### IPSS Risk Factor

### Low=7 (3%)

### Int-1=76 (27%)

### Int-2=128 (46%)

### High=57 (21%)

Study #2: Ph 3 Study AZA vs Conventional Care in MDS (Fenaux et al, Lancet Haematol 2009)

| | AZA | PRAC-AZA |
|---|---|---|
| | Monotherapy | Evaluable |
| | (N = 179) | (N = 60) |
| CR + PR | 29% (17 + 12) | 35% (33 + 2) |
| SD (include marrow CR) | 42% | 60% |
| HI | 49% | 69% |

Study #3: Dual epigenetic targeting with panobinostat and azacitidine in AML and high-risk MDS (Tan et al, Blood Cancer J 2014)

| | Pano-AZA | PRAC-AZA |
|---|---|---|
| | (N = 10) | (N = 60) |
| CR + PR + mCR | 5 (50%) | 73% |
| SD | 1 (10%) | 22% |
| PD | 3 (30%) | 5% |
| NE | 1 (10%) | |

Study #4: Prolonged Administration of Azacitidine With or Without Entinostat for MDS and AML With Myelodysplasia-Related Changes: Results of the US Leukemia Intergroup Trial E1905 (Prebet, J C O 2014)

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating high or very high risk myelodysplastic syndromes (MDS) in a patient in need thereof, the method comprising administering to the patient:
(i) a DNA hypomethylating agent; and
(ii) about 45 mg of a compound of formula (I): 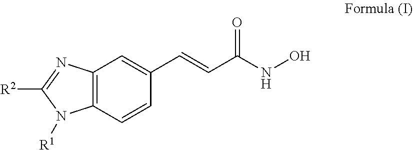
wherein
R¹ is —(CR²⁰R²¹)ₘ—(CR²²R²³)ₙ—(CR²⁴R²⁵)ₒ—NR²⁶R²⁷;
R² is alkyl, fluoroalkyl, cyano, C₂-C₆alkenyl, C₂-C₆alkynyl, or heteroalkyl optionally substituted with ═O;
each R²⁰, R²¹, R²², R²³, R²⁴, and R²⁵ is independently H or methyl;
each R²⁶ and R²⁷ is independently H, hydroxyalkyl, or alkyl; and
m, n, and o are independently integers of 0, 1, 2, 3, or 4;
or a pharmaceutically acceptable salt, isotopic variant, or prodrug thereof.

2. The method of claim 1, wherein the compound of formula (I) has the structure:

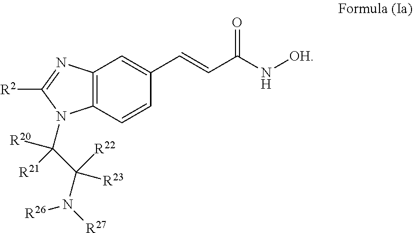

3. The method of claims 1 or 2, wherein R²⁶ and R²⁷ are independently H or alkyl.

4. The method of any one of claims 1-3, wherein R²⁶ and R²⁷ are independently H, methyl, ethyl, isopropyl, propyl, butyl, isobutyl, pentyl, hexyl or heptyl.

5. The method of any one of claims 1-4, wherein R¹ has the structure

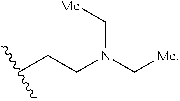 [SMILES: *CCN(CC)CC]

6. The method of any one of claims 1-5, wherein R² is ethyl, 1-methyl-ethyl, 2,2,2-trifluoroethyl, propyl, 2-methyl-propyl, 2,2-dimethyl-propyl, 3,3,3-trifluoro-propyl, butyl, 3,3-dimethyl-butyl, pentyl, 2,4,4-trimethyl-pentyl, hexyl or octyl.

7. The method of any one of claims 1-6, wherein R² is butyl.

8. The method of any one of claims 1-7, wherein the compound of formula (I) is pracinostat: 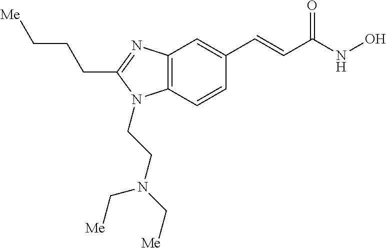 [SMILES: CCCCc1nc2cc(/C=C/C(=O)NO)ccc2n1CCN(CC)CC]
or a pharmaceutically acceptable salt, isotopic variant, or prodrug thereof.

9. The method of any one of claims 1-8, wherein the DNA hypomethylating agent is 5-azacytidine (azacitidine), 5-azadeoxycytidine (decitabine), SGI-110, zebularine, or procaine.

10. The method of any one of claims 1-9, wherein the DNA hypomethylating agent is 5-azacytidine (azacitidine).

11. The method of any one of claims 1-9, wherein the DNA hypomethylating agent is 5-azadeoxycytidine (decitabine).

12. The method of any one of claims 1-11, wherein the method is for treating high risk myelodysplastic syndromes (MDS).

13. The method of any one of claims 1-11, wherein the method is for treating very high risk myelodysplastic syndromes (MDS).

14. The method of any one of claims 1-13, wherein the patient in need thereof has not been previously treated with a DNA hypomethylating agent.

15. The method of any one of claims 1-14, wherein the patient in need thereof has been previously treated with transfusions, hematopoietic growth factors, or immunosuppressive therapy.

16. The method of any one of claims 1-13, wherein the MDS is refractory, non-responsive, or resistant to chemotherapy and/or haploidentical stem cell transplantation.

17. The method of any one of claims 1-16, wherein the DNA hypomethylating agent is administered in an amount from about 5 mg/m² to about 125 mg/m².

18. The method of any one of claims 1-17, wherein the DNA hypomethylating agent is administered in an amount of about 75 mg/m².

19. The method of any one of claims 1-18, wherein the compound of formula (I), or a pharmaceutically acceptable salt, isotopic variant, or prodrug thereof, is administered orally and the hypomethylating agent is administered intravenously or subcutaneously.

20. The method of any one of claims 1-19, wherein the compound of formula (I), or a pharmaceutically acceptable salt, isotopic variant, or prodrug thereof, and the DNA hypomethylating agent are administered in cycles of 28 days.

21. The method of any one of claims 1-20, wherein the compound of formula (I), or a pharmaceutically acceptable salt, isotopic variant, or prodrug thereof, and the DNA hypomethylating agent are administered for at least 3 cycles.

22. The method of any one of claims 1-20, wherein the compound of formula (I), or a pharmaceutically acceptable salt, isotopic variant, or prodrug thereof, and the DNA hypomethylating agent are administered for at least 4 cycles.

23. The method of any one of claims 1-20, wherein the compound of formula (I), or a pharmaceutically acceptable salt, isotopic variant, or prodrug thereof, and the DNA hypomethylating agent are administered for at least 5 cycles.

24. The method of any one of claims 1-20, wherein the compound of formula (I), or a pharmaceutically acceptable salt, isotopic variant, or prodrug thereof, and the DNA hypomethylating agent are administered for at least 6 cycles.

25. The method of any one of claims 1-20, wherein the compound of formula (I), or a pharmaceutically acceptable salt, isotopic variant, or prodrug thereof, and the DNA hypomethylating agent are administered for at least 7 cycles.

26. The method of any one of claims 1-20, wherein the compound of formula (I), or a pharmaceutically acceptable salt, isotopic variant, or prodrug thereof, and the DNA hypomethylating agent are administered for at least 8 cycles.

27. The method of any one of claims 1-20, wherein the compound of formula (I), or a pharmaceutically acceptable salt, isotopic variant, or prodrug thereof, and the DNA hypomethylating agent are administered for at least 9 cycles.

28. The method of any one of claims 1-20, wherein the compound of formula (I), or a pharmaceutically acceptable salt, isotopic variant, or prodrug thereof, and the DNA hypomethylating agent are administered for at least 10 cycles.

29. The method of any one of claims 1-20, wherein the compound of formula (I), or a pharmaceutically acceptable salt, isotopic variant, or prodrug thereof, and the DNA hypomethylating agent are administered until complete remission (CR) is observed.

30. The method of any one of claims 1-29, wherein the compound of formula (I), or a pharmaceutically acceptable salt, isotopic variant, or prodrug thereof, is administered for 3 days each week for 3 consecutive weeks, followed by 1 week of rest of each 28-day cycle.

31. The method of any one of claims 1-30, further comprising administering the compound of formula (I), or a pharmaceutically acceptable salt, isotopic variant, or prodrug thereof, for 3 days each week for 2 consecutive weeks, followed by 2 weeks of rest of each 28-day cycle.

32. The method of any one of claims 1-31, wherein the DNA hypomethylating agent is administered for 7 days of each 28-day cycle.

33. The method of any one of claims 1-31, wherein the DNA hypomethylating agent is administered on a 5-2-2 schedule: DNA hypomethylating agent for 5 consecutive days followed by 2 days of rest, followed by DNA hypomethylating agent for 2 consecutive days of each 28-day cycle.

34. The method of any one of claims 1-33, wherein the discontinuation rate due to adverse events is less than 25%, less than 20%, less than 15%, less than 10%, less than 8%, less than 5%.

35. The method of any one of claims 1-33, wherein the discontinuation rate due to adverse events is about 4%.

36. The method of claim 34 or 35, wherein the adverse event is selected from constipation, nausea, fatigue, decreased appetite, diarrhea, edema peripheral, hypoalbuminemia, dyspnea, hypokalemia, vomiting, dizziness, febrile neutropenia, anemia, neutropenia, and thrombocytopenia.

37. A method of treating high or very high risk myelodysplastic syndromes (MDS) in a patient in need thereof, the method comprising administering to the patient:
(i) a DNA hypomethylating agent; and
(ii) about 45 mg of pracinostat, or a pharmaceutically acceptable salt, isotopic variant, or prodrug thereof, wherein pracinostat is orally administered to the patient 3 days each week for 3 consecutive weeks, followed by 1 week of rest, in 28-day cycles.

38. The method of claim 37, wherein the DNA hypomethylating agent and pracinostat or a pharmaceutically acceptable salt, isotopic variant, or prodrug thereof, are administered for at least 3 cycles.

39. The method of claim 37, wherein the DNA hypomethylating agent and pracinostat or a pharmaceutically acceptable salt, isotopic variant, or prodrug thereof, are administered for at least 4 cycles.

40. The method of claim 37, wherein the DNA hypomethylating agent and pracinostat or a pharmaceutically acceptable salt, isotopic variant, or prodrug thereof, are administered for at least 5 cycles.

41. The method of claim 37, wherein the DNA hypomethylating agent and pracinostat or a pharmaceutically acceptable salt, isotopic variant, or prodrug thereof, are administered for at least 6 cycles.

42. The method of claim 38, wherein the DNA hypomethylating agent and pracinostat or a pharmaceutically acceptable salt, isotopic variant, or prodrug thereof, are administered for at least 3 cycles, followed by further administering pracinostat, or a pharmaceutically acceptable salt, isotopic variant, or prodrug thereof, for 3 days each week for 2 consecutive weeks, followed by 2 weeks of rest of each 28-day cycle for at least 1 cycle.

43. A method of treating high or very high risk myelodysplastic syndromes (MDS) in a patient in need thereof, the method comprising administering to the patient:
(i) about 75 mg/m² of 5-azacytidine (azacitidine), wherein 5-azacytidine (azacitidine) is administered intravenously or subcutaneously for 7 days of each 28-day cycle; and
(ii) about 45 mg of pracinostat, or a pharmaceutically acceptable salt, isotopic variant, or prodrug thereof, wherein pracinostat is orally administered for 3 days each week for 3 consecutive weeks, followed by 1 week of rest, in 28-day cycles.

44. The method of claim 43, wherein 5-azacytidine (azacitidine) and pracinostat or a pharmaceutically acceptable salt, isotopic variant, or prodrug thereof, are administered for at least 3 cycles.

45. The method of claim 43, wherein 5-azacytidine (azacitidine) and pracinostat or a pharmaceutically acceptable salt, isotopic variant, or prodrug thereof, are administered for at least 4 cycles.

46. The method of claim 43, wherein 5-azacytidine (azacitidine) and pracinostat or a pharmaceutically acceptable salt, isotopic variant, or prodrug thereof, are administered for at least 5 cycles.

47. The method of claim 43, wherein 5-azacytidine (azacitidine) and pracinostat or a pharmaceutically acceptable salt, isotopic variant, or prodrug thereof, are administered for at least 6 cycles.

48. The method of claim 44, wherein 5-azacytidine (azacitidine) and pracinostat or a pharmaceutically acceptable salt, isotopic variant, or prodrug thereof, are administered for at least 3 cycles, followed by further administering pracinostat, or a pharmaceutically acceptable salt, isotopic variant, or prodrug thereof, for 3 days each week for 2 consecutive weeks, followed by 2 weeks of rest of each 28-day cycle for at least 1 cycle.
